(19) United States
(12) Patent Application Publication
(10) Pub. No.: US 2026/0345924 A1
(43) Pub. Date: Nov. 5, 2026

(54) INHALATION DEVICE WITH INTEGRAL PISTON

(71) Applicant: invoX Belgium NV, Diepenbeek (BE)

(72) Inventors: Juergen Rawert, Koeln (DE); Steven Madden, Hamburg (DE); Steven Dudley, Brighton (GB)

(21) Appl. No.: 19/521,380

(22) PCT Filed: Oct. 4, 2024
(86) PCT No.: PCT/EP2024/077931

§ 371 Date: Mar. 20, 2026

(30) Foreign Application Priority Data
Oct. 4, 2023 (EP) 23201573.5
Oct. 4, 2023 (EP) 23201574.3

(51) Int. Cl. A61M 11/00; A61M 15/00

(52) U.S. Cl. CPC A61M 11/007; A61M 15/009

(57) ABSTRACT

The invention provides a hand-held inhalation device for delivering a medically active fluid (F) in aerosolized (nebulized) form for inhalation therapy, comprising (a) a housing (1) having a user-facing side; (b) a nozzle (6) for generating the nebulized aerosol by collision of at least two liquid jets, the nozzle (6) being firmly affixed to the user-facing side of the housing (1) such as to be immobile relative to the housing (1); (c) a fluid reservoir (2) arranged within the housing (1) for holding the medically active fluid; and (d) a pumping unit arranged within the housing (1) whereas the pumping unit comprises a riser pipe and a holder in the form of a one-piece unit.

### FIELD OF THE INVENTION

The invention relates to the field of inhalation device for delivering a medically active fluid in aerosolized form for inhalation therapy. In particular, the invention relates to a hand-held inhalation device comprising a pumping unit having a riser pipe and a corresponding holder in the form of a one-piece unit as well as to a corresponding method.

### BACKGROUND OF THE INVENTION

Pumping devices or units for medical devices, specifically for pumping medical fluids are known from the art since a long time ago. For example, such devices are known from inhalation devices for the application of medical fluids or active ingredients in the form of aerosols, i.e. small liquid droplets embedded in a gas. Such an inhalation device is known e.g. from document WO 91/14468 A1. Essential components of this inhalation device are a reservoir in which the liquid that is to be aerosolized is contained; a pumping device for generation of a pressure being sufficiently high for nebulizing; as well as an atomizing device in the form of a nozzle. By means of the pumping device, the liquid is drawn in a discrete amount, i.e. not continuously, from the reservoir, and fed to the nozzle. The pumping device works without propellant and generates pressure mechanically.

The document further discloses an embodiment in which the pressure in the pumping chamber which is connected to the housing is generated by movement of a moveable hollow piston. The piston is moveably arranged inside the immobile cylinder or pumping chamber. The (upstream arranged) inlet of the hollow piston is fluidically connected to the interior of the reservoir (reservoir pipe section). Its (downstream arranged) tip leads into the pumping chamber. Furthermore, a check valve that inhibits a back flow of liquid into the reservoir is arranged inside the tip of the piston.

For filling the piston, the same is directly connected with its upstream end to the reservoir. By pulling out the piston of the pumping chamber provided inside a hollow cylinder, its interior volume is enlarged, such that an increasing under pressure is built up inside the pumping chamber. This pressure propagates through the hollow piston into the reservoir, such that liquid is sucked from the same into the piston. At the same time, said valve opens at its tip, since the pressure inside the reservoir is higher than inside the (yet empty) pumping chamber. The pumping chamber is being filled. At the same time, a spring is loaded, and locked at the motion's end when the moveable piston has reached its lower dead center (in the case of a vertically arranged device) and the pumping chamber is filled.

A further example of a pumping device for the generation of discrete amounts of a medical fluid in pressurized form for inhalation is disclosed in US 2014/0076308 A1. The document discloses a specific inhaler having a pumping unit with a pressure chamber and a conveying tube acting as a piston within the pressure chamber. The conveying tube has nonreturn valve positioned at its the downstream end located within the pressure chamber.

The piston is arranged inside the pressure spring which is designed as a helical spring, thus limiting its outside diameter. Also because of the typically small volume (e.g. 15 μl), the piston is designed with a thin interior (and often also exterior) diameter.

This typically small inner diameter of the conveying tube or, in other words, moveable piston of e.g. 0.3 to 1.0 mm, together with a small size of the check valve being arranged within, is a drawback of the described construction. The small diameter results in a high flow resistance, such that in particular, media of higher viscosities flow into and through the piston only very slowly. In other words, the described construction is suitable especially for low-viscosity (aqueous) liquids and for emitting low doses thereof. Furthermore, fabrication of a sufficiently tight check valve of small diameter is difficult.

An improved inhalation device utilizing a non-moveable piston is disclosed in WO 2018/197730 A1. The device overcomes some of the drawbacks of the above-described prior art.

Furthermore, the pumping units of the above-described devices are designed for generating discrete portions of the medical fluid to be aerosolized at elevated pressures necessary for the aerosolization of the respective medical fluid, often in the range of about 10 to about 100 bar and above. Accordingly, especially the components of the corresponding pumping units, specifically the corresponding pistons for the generation of the pressure are manufactured from a mechanically stable and pressure-inert material, typically from a metal such as stainless steel.

This, however, especially in connection with the spatial restraints as discussed above results in the need for providing precisely machined metallic components which significantly contribute to the overall manufacturing costs of the respective devices which is especially problematic for devices with a limited number of uses.

It is thus an object of the present invention to provide for an improved high-pressure medical pumping device for providing discrete portions of a medical fluid in pressurized form that overcomes the at least one of the above-discussed drawbacks of the prior art devices. Further objects of the invention will be clear on the basis of the following description of the invention, examples and claims.

### SUMMARY OF THE INVENTION

In a first aspect, the invention relates to a hand-held inhalation device for delivering a medically active fluid (F) in aerosolized form for inhalation therapy, comprising
(a) a housing (1) having a user-facing side;
(b) a nozzle (6) for generating the nebulized aerosol by collision of at least two liquid jets, the nozzle (6) being firmly affixed to the user-facing side of the housing (1) such as to be immobile relative to the housing (1);
(c) a fluid reservoir (2) arranged within the housing (1) for holding the medically active fluid; and
(d) a pumping unit arranged within the housing (1), the pumping unit having
an upstream end that is fluidically connected to the fluid reservoir (2); and
a downstream end that is fluidically connected to the nozzle (6);
   wherein the pumping unit is adapted for pumping the medically active fluid (F) from the fluid reservoir (2) to the nozzle (6); wherein the pumping unit further comprises
(i) a riser pipe (5) having an upstream end and a downstream end, wherein the riser pipe (5) is adapted to function as a piston in the pumping unit;
   (ii) a holder (10) located downstream of the riser pipe being firmly affixed to the user-facing side of the housing (1) such as to be immobile relative to the housing (1) for holding the riser pipe;
   (iii) a hollow cylinder (9) located upstream of the riser pipe (5), wherein the upstream end of the riser pipe (5) is inserted in the cylinder (9) such that the cylinder (9) is longitudinally movable on the riser pipe (5); and
   (iv) a lockable means for storing potential energy (7) when locked and for releasing the stored energy when unlocked, the means (7) being arranged outside of, and mechanically coupled to, the cylinder (9) such that unlocking the means (7) results in a propulsive longitudinal movement of the cylinder (9) towards the downstream end of the pumping unit;
wherein the holder (10) and the riser pipe (5) are a one-piece unit.

In a particular embodiment of the first aspect the invention provides a hand-held inhalation device for delivering a medically active fluid (F) in aerosolized form for inhalation therapy, comprising
(a) a housing (1) having a user-facing side, the housing further comprises a receptacle configured for attaching a riser pipe (5);
(b) a nozzle (6) for generating the nebulized aerosol by collision of at least two liquid jets, the nozzle (6) being firmly affixed to the user-facing side of the housing (1) such as to be immobile relative to the housing (1);
(c) a fluid reservoir (2) arranged within the housing (1) for holding the medically active fluid; and
(d) a pumping unit arranged within the housing (1), the pumping unit having
an upstream end that is fluidically connected to the fluid reservoir (2); and
a downstream end that is fluidically connected to the nozzle (6);
   wherein the pumping unit is adapted for pumping the medically active fluid (F) from the fluid reservoir (2) to the nozzle (6); wherein the pumping unit further comprises
(i) the riser pipe (5) monolithically comprising:
   a riser pipe segment having an upstream end and a downstream end
   a holder segment (10) located at the downstream end of the riser pipe segment, the holder segment attached to the receptacle of the housing and firmly affixing the riser pipe (5) to the user-facing side of the housing (1) such as to be immobile relative to the housing (1);
      (ii) a hollow cylinder (9) located upstream of the riser pipe (5), wherein the upstream end of the riser pipe (5) is inserted in the cylinder (9) such that the cylinder (9) is longitudinally movable on the riser pipe segment; and
(iii) a lockable means for storing potential energy (7) when locked and for releasing the stored energy when unlocked, the means (7) being arranged outside of, and mechanically coupled to, the cylinder (9) such that unlocking the means (7) results in a propulsive longitudinal movement of the cylinder (9) towards the downstream end of the pumping unit.

In a second aspect, the present invention provides for a one-piece unit comprising a riser pipe and a holder adapted to be comprised by the inhalation device according to the first aspect of the invention.

In a third aspect, the invention provides for a method for the manufacture of a one-piece according to the second aspect of the invention, wherein the method comprises injection-molding of an injection-moldable material.

In a fourth aspect, the invention provides for a method for the generation of a nebulized aerosol of a medically active liquid. The method may involve the use of the inhalation device of the first aspect of the invention as described herein-above, in any of its embodiments.

### BRIEF DESCRIPTION OF THE DRAWINGS

FIG. 1 shows schematically an embodiment of an inhalation device according to the invention prior to its first use.

FIG. 2 shows a device similar to the one of FIG. 1, but without an outlet valve.

FIG. 3 shows the embodiment of FIG. 1 with a filled pumping chamber.

FIG. 4 shows the situation during the first actuation of the device.

FIG. 5 shows the situation at the end of the first actuation.

FIG. 6 shows the situation after re-filling the pumping chamber.

FIG. 7 depicts a further embodiment of an inhalation device according to the present invention comprising a riser pipe and a holder provided as a one-piece structure.

FIGS. 8 to 10 depict one embodiment of a high-pressure medical pumping device for providing discrete portions of a medical fluid (F) in pressurized form.

FIGS. 11 to 13 depict an alternative embodiment of a high-pressure medical pumping device for providing discrete portions of a medical fluid (F) in pressurized form.

### DETAILED DESCRIPTION OF THE INVENTION

The following terms or expressions as used herein should normally be interpreted as outlined in this section, unless defined otherwise by the description or unless the specific context indicates or requires otherwise:

The words ‘comprise’, ‘comprises’ and ‘comprising’ and similar expressions are to be construed in an open and inclusive sense, as ‘including, but not limited to’ in this description and in the claims. In contrast to this, the terms “consist of”, “consists of” and “consisting of” as used herein are so-called closed language meaning that only the mentioned components are present.

The terms ‘a’ or ‘an’ do not exclude a plurality; i.e. the singular forms ‘a’, ‘an’ and ‘the’ should be understood as to include plural referents unless the context clearly indicates or requires otherwise. In other words, all references to singular characteristics or limitations of the present disclosure shall include the corresponding plural characteristic or limitation, and vice versa, unless explicitly specified otherwise or clearly implied to the contrary by the context in which the reference is made. The terms ‘a’, ‘an’ and ‘the’ hence have the same meaning as ‘at least one’or as ‘one or more’unless defined otherwise.

The expressions, ‘one embodiment’, ‘an embodiment’, ‘a specific embodiment’ and the like mean that a particular feature, property or characteristic, or a particular group or combination of features, properties or characteristics, as referred to in combination with the respective expression, is present in at least one of the embodiments of the invention. The occurrence of these expressions in various places throughout this description do not necessarily refer to the same embodiment. Moreover, the particular features, properties or characteristics may be combined in any suitable manner in one or more embodiments.

The terms ‘essentially’, ‘about’, ‘approximately’, ‘substantially” and the like in connection with an attribute or value include the exact attribute or the precise value, as well as any attribute or value typically considered to fall within a normal range or variability accepted in the technical field concerned. For example, the term 'about’ in connection with a value or range of values as used herein shall mean that such value or range of values includes typical deviations from such values of up to +/−5% (abs.), or up to about +/−4%, or up to +/−3% up to +/−2%, up to +/−1%, up to +/−0.5%.

The present invention provides a hand-held inhalation device for delivering a nebulized medically active fluid in aerosolized or, in other words, nebulized form for inhalation therapy according to the first aspect of the invention.

More specifically, the device comprises a housing having a user-facing side, a nozzle, specifically an impingement-type nozzle as described further below for generating the nebulized aerosol by collision of at least two liquid jets, a fluid reservoir for holding the medically active fluid arranged within the housing, and a pumping unit which is also arranged within the housing. The nozzle is firmly affixed or, in other words, attached to the user-facing side of the housing such as to be immobile relative to the housing. The pumping unit has an upstream end that is fluidically connected to the fluid reservoir and a downstream end that is fluidically connected to the nozzle. Furthermore, the pumping unit is adapted for pumping the medically active fluid from the fluid reservoir to the nozzle, and it comprises a riser pipe which is adapted to function as a piston in the pumping unit, a holder for holding the riser pipe, said holder being located downstream of the riser pipe or, more specifically, at the downstream end of the riser pipe, and being firmly affixed to the user-facing side of the housing such as to be immobile relative to the housing. The pumping unit further comprises a hollow cylinder and a lockable means for storing potential energy.

The riser pipe is firmly affixed to the user-facing side of the housing such as to be immobile relative to the housing via the holder. In the context of the present invention the term “holder” refers to a structural element integrated with the downstream end of the riser pipe, which interfaces with other features of the hand-held inhalation device, such as for attaching to the housing. Thus, the holder provides a stable and fixed position for the riser pipe relative to the other components of the hand-held inhalation device. In general, the holder is wider than the diameter of the riser pipe and preferably comprises features for attachment and/or fixing to other parts of the inhalation device.

The hollow cylinder is located upstream of the riser pipe, and the upstream end of the riser pipe is inserted in the cylinder such that the cylinder is longitudinally movable on the riser pipe. The lockable means is capable of storing potential energy when locked and adapted for releasing the stored energy when unlocked. The means is arranged outside of, and mechanically coupled to, the cylinder in such a way that unlocking the means results in a propulsive longitudinal movement of the cylinder towards the downstream end of the pumping unit. According to the invention, the holder and the riser pipe are a one-piece unit or, in other words, are an integral one-piece unit, or, in other words, are provided in a form in which they cannot be separated from each other in a reversible or non-destructive manner. Accordingly, the holder for holding the riser pipe and for fixing the riser pipe relative to the rest of the pumping unit and inhalation device is in the form of a one-piece unit in which the riser pipe and the holder cannot be detached from each other in a non-destructive and reversible manner.

As used herein, a hand-held inhalation device is a mobile device which can be conveniently held in one hand and which is suitable for delivering a nebulized medically active aerosol for inhalation therapy. In order to be suitable for inhalation therapy, the device must be able to emit a medically active aerosol whose particle size is respirable, i.e. small enough to be taken up by the lungs of a patient or user.

Typically, respirable particles have a mass median aerodynamic diameter of not more than about 10 μm, in particular not more than about 7 μm, or not more than about 5 μm, respectively. In this respect, inhalation devices are substantially different from devices that emits spray for oral or nasal administration, such as disclosed in US 2004/0068222 A1.

According to the invention, the inhalation device is capable of delivering a nebulized aerosol, specifically for delivering the medically active fluid in aerosolized form. As used herein, an aerosol is a system having at least two phases: a continuous phase which is gaseous and which comprises a dispersed liquid phase in the form of small liquid droplets. Optionally, the liquid phase may itself represent a liquid solution, dispersion, suspension, or emulsion.

The term “medically active fluid” as used herein is to be interpreted broadly and means a fluid or preferably a liquid that may be applied or administered to a subject, for example by inhalation, and that preferably is physiologically and/or pharmaceutically acceptable. In some embodiments, the medically active fluid may be water or another physiologically and/or pharmaceutically acceptable liquid compound or mixture of liquid compounds or a liquid composition comprising one or more physiologically and/or pharmaceutically acceptable ingredients liquid such as a solution or dispersion. In some embodiments, the medically active fluid may be a liquid composition comprising an aqueous or alcoholic liquid solvent or, in other words, vehicle and optionally at least one medically active ingredient and optionally at least one pharmaceutically acceptable excipient wherein the term “excipient” as used herein means a compound which may be comprised by the medically active fluid including, but are not limited to, one or more buffering agents to regulate or control pH of the solution such as hydrochloric acid, chelating agents such as EDTA, salts such as sodium chloride, taste-masking agents, surfactants, lipids, antioxidants, and co-solvents, which may be used to enhance or improve solubility as well as preservatives such as benzalkonium chloride.

Suitable excipients are known to the skilled person and are described, e.g. in standard pharmacopoeias such as U.S.P. or Ph. Eur., or in the Handbook of Pharmaceutical Excipients, 6th ed. Rowe et al, Eds.; The Pharmaceutical Press and the American Pharmaceutical Association: 2009.

In some embodiments, the medically active fluid may simply be water or an aqueous solution of sodium chloride. In further embodiments, the medically active fluid may comprise at least one active pharmaceutical ingredient, for example selected from the groups of long acting muscarinic antagonists (LAMAs), for example tiotropium, aclidinium, umeclidinium and glycopyrrolium, or long acting beta antagonists (LABAs) for example olodaterol, vilanterol, or inhalable glucocorticoids (ICSs) such as predinisolone, ciclesonide, beclomethasone and others.

Important for the generation of a nebulized aerosol is a suitable nozzle. In some embodiments of the present inhalation device, the nozzle is of the impingement type. This means that the nozzle is adapted to emit at least two jets of liquid which are directed such as to collide and break up into small aerosol droplets. The nozzle is firmly affixed to the user-facing side of the housing of the inhalation device in such a way that it is immobile, or non-moveable, relative to the housing or at least relative to the side or part of the housing which faces the user (e.g. patient) when the device is used.

The fluid reservoir which is arranged within the housing is adapted to hold or store the medically active fluid or liquid from which the nebulized aerosol is generated and delivered by the inhalation device.

The pumping unit which is also arranged within the housing is adapted to function as a piston pump, also referred to as plunger pump, wherein the riser pipe functions as the piston, or plunger, which is longitudinally moveable within the hollow cylinder.

The inner segment of the hollow cylinder in which the upstream end of the riser pipe moves forms a pumping chamber which has a variable volume, depending on the position of the riser pipe relative to the cylinder.

The hollow cylinder which provides the pumping chamber is fluidically connected with the fluid reservoir, either directly or indirectly, such as by means of an optional reservoir pipe (or reservoir pipe section). Similarly, the riser pipe, whose reservoir-facing, interior (upstream) end which can be received in the hollow cylinder, is fluidically connected at its downstream or exterior end to the nozzle in a liquid-tight manner, either directly or indirectly.

In this context, the expression “hollow cylinder” refers to a part or member which is hollow in the sense that it comprises an internal void which has a cylindrical shape, or which has a segment having a cylindrical space. In other words, and as is applicable to other types of piston pumps, it is not required that the external shape of the respective part or member is cylindrical. Moreover, the expression “hollow cylinder” does not exclude an operational state of the respective part or member in which the “hollow” space may be filled with material, e.g. with a liquid to be nebulized.

As used herein, a longitudinal movement means a movement along the main axis of the hollow cylinder, and a propulsive movement is a movement of a part in a downstream (or forward) direction.

Importantly, the riser pipe of the pumping unit of the inhalation device of the invention is arranged downstream of the cylinder, and it is firmly affixed to the user-facing side of the housing such as to be immobile relative to the housing or at least to the part of the housing which comprises the user-facing side of the housing via a holder as described further below. For the avoidance of doubt, the term ‘firmly fixed’ means either directly or indirectly (i.e. via one or more connecting parts) fixed such as to prevent relative movement between the respective parts. As the nozzle is also immobile relative to the housing or the respective part of the housing, the riser pipe is also immobile relative to the nozzle, and the pumping action is effected by the longitudinal movement of the hollow cylinder. A propulsive movement of the cylinder, which is arranged in an upstream position relative to the riser pipe, results in a decrease of the volume of the pumping chamber, and a repulsive movement of the cylinder results in an increase of the volume. In other words, the riser pipe maintains its position relative to the housing, and the hollow cylinder can alter its position relative to the housing, and in particular, along a longitudinal axis of the same, such as to perform a piston-in-cylinder-type movement of the immobile riser pipe in the moveable cylindrical member.

This arrangement differs from other impingement-type inhalation devices which rely on a pumping unit whose riser pipe is in an upstream position and a cylindrical member in a downstream position wherein the riser pipe is moveable and the cylindrical member is fixed to the housing, as disclosed in US 2012/0090603 A1. A key advantage of the design of the present device is that the passage between pumping chamber and fluid reservoir can be designed with less restrictions with respect to its dimensions. It is e.g. possible to accommodate a significantly larger inlet valve (also referred to as check valve), which is easier to manufacture since it does not have to be contained within a narrow riser pipe. Instead, the design of the present inhalation device allows the use of a check valve whose size is only restricted by the interior size of the housing or the dimensions of the means for storing potential energy. In other words, the diameters of the valve, the riser pipe and -if used-the reservoir pipe do not need to match each other. Furthermore, since no movable piston needs to be connected to the fluid reservoir, the component which provides the fluid connection to the reservoir can be designed independently of the moveable component, i.e. the hollow cylinder, allowing the individual parts to be adapted to suit their respective individual functions. In this respect, the design of the present device provides for higher design flexibility because the moveable hollow cylinder, due to its robust structure and dimensions, provides better opportunities for designing a mechanically stable connection with the reservoir than would a less robust moveable riser pipe. Also, the connection between the hollow cylinder and the fluid reservoir can be designed with a larger diameter, such that higher flow velocities and fluid viscosities become feasible. Further, a support for the reservoir can be integrated into any component that comprises the cylinder. Additionally, any vent for pressure equilibration of the reservoir can be moved away from the reservoir body itself to (e.g.) a connector which forms an interface between reservoir and hollow cylinder, thus facilitating construction and avoiding the necessity to provide an essentially “open” reservoir body.

As mentioned, the lockable means for storing potential energy is adapted to store energy in its locked state and to release the stored energy when unlocked. The means is mechanically coupled to the hollow cylinder in such a way such that unlocking the means results in a propulsive longitudinal movement of the cylinder towards the downstream end of the pumping unit. During this movement, the internal volume of the cylinder, i.e. the volume of the pumping chamber, decreases. Vice versa, when the means for storing potential energy is in the locked state, the hollow cylinder is in its most upstream position in which the volume of the pumping chamber is largest. The locked state could also be considered a primed state. When the state of the means for storing energy is altered from the unlocked to the locked state, which could be referred to as priming the device, the hollow cylinder performs a repulsive longitudinal movement, i.e. from its most downstream position towards its most upstream position. A pumping cycle consists of two subsequent and opposing movements of the cylinder starting from its most downstream position to its most upstream (or primed) position and—driven by the means for storing potential energy that now releases its energy—back to its most downstream position.

In one of the preferred embodiments, the pumping unit is a high-pressure pumping unit and adapted to operate, or to expel fluid, at a pressure of at least about 50 bar. In other preferred embodiments, the operating pressure of the pumping unit is at least about 10 bar, or at least about 100 bar, or from about 2 bar to about 1000 bar, or from about 50 bar to about 250 bar, respectively. In some embodiments, the operating pressure may be in the range of at least about 150 bar or of at least about 175 or of at least about 200 bar, for example from about 100 bar to about 1000 bar or even higher, or from at least about 125 bar, for example from about 125 bar to about 750 bar or from at least about 150 bar, for example from about 150 bar to about 500 bar, or from at least about 175 bar, for example from about 175 bar to about 400 bar, or from at least about 200 bar, for example from about 200 bar to about 300 bar, or from about 100 bar or from about 150 bar to about 300 bar. As used herein, the operating pressure is the pressure at which the pumping unit expels fluid, in particular a medically active fluid or liquid, such as an inhalable aqueous liquid formulation of a pharmacologically active ingredient, from its pumping chamber in a downstream direction, i.e. towards the nozzle. In this context, the expression “adapted to operate” means that the components of the pumping unit are selected with respect to the materials, the dimensions, the quality of the surfaces and the finish are selected such as to enable operation at the specified pressure.

Moreover, such high-pressure pumping unit implies that the means for storing potential energy is capable of storing and releasing a sufficient amount of energy to drive the propulsive longitudinal movement of the cylinder with such a force that the respective pressure is obtained.

The means for the storage of potential energy may be designed as tension or pressure spring. Alternatively, besides a metallic or plastic body, also a gaseous medium, or magnetic force utilizing material can be used as means for energy storage. By compressing or tensioning, potential energy is fed to the means. One end of the means may be supported at or in the housing at a suitable location; thus, this end may be essentially immobile. With the other end, it may be connected to the hollow cylinder which provides the pumping chamber; thus, this end may be essentially moveable.

The means can be locked after being loaded with a sufficient amount of energy, such that the energy can be stored until unlocking takes place. When unlocked, the means can release the potential energy (e.g. spring energy) to the cylinder with the pumping chamber, which is then driven such as to perform a (in this case, longitudinal) movement. Typically, the energy release takes place abruptly, so that a high pressure can build up inside the pumping chamber before a significant amount of liquid is emitted, which results in a pressure decrease. In fact, during a significant portion of the ejection phase, an equilibrium exists of pressure delivered by the means for the storage of potential energy, and the amount of already emitted liquid. Thus, the amount of liquid remains essentially constant during this phase, which is a significant advantage to devices which use manual force of the user for the emission, such as the devices disclosed in documents US 2005/0039738 A1, US 2009/0216183 A1, US 2004/0068222 A1, or US 2012/0298694 A1, since manual force depends on the individual user or patient and is very likely to vary largely during the ejection phase, resulting in inhomogeneous droplet formation, size, and amount. In contrast to the prior art, the means according to the invention ensures that the inhalation device delivers highly reproducible results.

The means for storing potential energy may also be provided in the form of a highly pressurized gas container. By suitable arrangement and repeatable intermittent activating (opening) of the same, part of the energy which is stored inside the gas container can be released to the cylinder. This process can be repeated until the remaining energy is insufficient for once again building up a desired pressure in the pumping chamber. After this, the gas container must be refilled or exchanged.

In one of the preferred embodiments, the means for storing potential energy is a spring having a load of at least 10 N in a deflected state. In a particularly preferred embodiment, the means for storing potential energy is a compression spring made of steel having a load from about 1 N to about 500 N in its deflected state. In other preferred embodiments, the compression spring from steel has a load from about 2 N to about 200 N, or from about 10 N to about 100 N, in its deflected state. In particular embodiments, such as a spring may have a load of at least about 30 N or at least about 35 N or at least about 40 N or selected within a range of from about 50 N to about 200 N or to about 150 N or to about 100N in a deflected state or selected in the range of from about 30 N to about 150 N or to about 100 N, or selected within the range of from about 35 N to about 80 N in a deflected state. In further embodiments, the pumping unit may further comprise a lock for locking the lockable means. This may allow the driving means to be tensioned or loaded and, in a separate step, to be triggered by unlocking the lockable means for storing potential energy to drive the pumping unit.

The inhalation device according to the invention is preferably adapted to deliver the nebulized medically active aerosol in a discontinuous manner, i.e. in the form of discrete units, wherein one unit is delivered per pumping cycle. In this aspect, the device differs from commonly known nebulizers such as jet nebulizers, ultrasonic nebulizers, vibrating mesh nebulizers, or electrohydrodynamic nebulizers which typically generate and deliver a nebulized aerosol continuously over a period of several seconds up to several minutes, such that the aerosol requires a number of consecutive breathing maneuvers in order to be inhaled by the patient or user. Instead, the inhalation device of the invention is adapted to generate and emit discrete units of aerosol, wherein each of the units corresponds to the amount (i.e. volume) of fluid (i.e. medically active fluid or liquid) which is pumped by the pumping unit in one pumping cycle into the nozzle where it is immediately aerosolized and delivered to the user or patient. Vice versa, the amount of liquid pumped by the pumping unit in one pumping cycle determines the amount of the pharmacologically active agent which the patient receives per dosing. It is therefore highly important with respect to achieving the desired therapeutic effect that the pumping unit operates precisely, reliably and reproducibly.

In one preferred embodiment, a single dose of the medication (i.e. of the nebulized aerosol of the medically active fluid or liquid) is contained in one unit, i.e. in the volume that is delivered from the pumping unit to the nozzle for aerosol generation in one single pumping cycle. In this case, the user or patient will prime and actuate the device only once, and inhale the released aerosol in one breathing maneuver, per dosing (i.e. per dosing event).

In another preferred embodiment, a single dose of the medication consists of two units of the aerosol, and thus requires two pumping cycles. Typically, the user or patient will prime the device, actuate it such as to release and inhale a unit of the aerosol, and then repeat the procedure. Alternatively, three or more aerosol units may constitute a single dosing.

The volume of fluid (e.g. of medically active liquid) that is pumped by the pumping unit in one pumping cycle is preferably in the range from about 2 to about 150 μl. In particular, the volume may range from about 0.1 μL to about 1000 μl, or from about 1 μL to about 250 μl, or from about 1 μL to about 250 μl, or from about 2 μL to about 150 μL, or from about 5 μL to about 100 μL, or from about 10 μL to about 75 μL, or from about 15 μL to about 50 μL, or from about 20 μL to about 40 μL or to about 30 μL, or from about 10 μL to about 30 μL or to about 20 μL, respectively. This volume ranges are nearly the same as the volume of liquid phase that is contained in one unit of aerosol generated by the inhalation device, perhaps with minor differences due to minute losses of liquid in the device.

In another preferred embodiment, the pumping unit comprises an inlet valve, also referred to as a check valve or inlet check valve, positioned in or adjacent to the hollow cylinder. According to this embodiment, the interior space of the hollow cylinder, i.e. the pumping chamber, is fluidically connected with the fluid reservoir via the inlet check valve. The inlet valve allows the inflow of liquid into the pumping chamber, but prevents the backflow of liquid towards, or into, the fluid reservoir. The position of the inlet valve may be at or near the upstream end of the cylinder such as to make nearly the entire internal volume of the hollow cylinder available for functioning as the pumping chamber. Alternatively, it may be more centrally located along the (longitudinal) main axis of the hollow cylinder such as to define an upstream segment and a downstream segment of the cylinder, the upstream segment being upstream of the inlet valve and the downstream segment being downstream of the valve. In this case the pumping chamber is located in the downstream segment.

As mentioned, one of the advantageous effects of the design of the present inhalation device is that an inlet valve having relatively large dimensions may be accommodated in this position, i.e. at the upstream end of the pumping chamber. This is particularly beneficial as it allows for large dimensions of the fluid conduit(s) within the valve, thus enabling high fluid velocities which translate into a rapid filling of the pumping chamber during the priming of the inhalation device. Moreover, the use of liquids having a higher viscosity than ordinary liquid formulations for inhalation, such as highly concentrated solutions of soluble active ingredients, become feasible for inhalation therapy.

According to some embodiments, the inlet valve may be adapted to open only when the pressure difference between the upstream and the downstream side of the valve, i.e. the fluid reservoir side and the pumping chamber side, is above a predefined threshold value, and remains closed as long as the pressure difference is below the threshold value. The term “pressure difference” as used in this regard means that, irrespective of the absolute pressure values, only the relative pressure difference between the two sides is relevant for determining whether the valve blocks or opens. If, for example, the pressure on the upstream (reservoir) side is already positive (e.g. 1.01 bar due to thermal expansion), but the pressure on the downstream (pumping chamber) side is ambient pressure (1.0 bar, no activation of the device), the pressure difference (here: 0.01 bar) is below the threshold value (e.g. 20 mbar), which allows the valve to stay closed even when subject to a positive pressure in opening direction. This means that the check valve remains closed until the threshold pressure is met, thus keeping the passage between reservoir and pumping chamber safely shut e.g.

when the inhalation device is not in use. Examples for threshold pressure differences are in the range of 1 to 1000 mbar, and more preferably between about 10 and about 500 mbar, or between about 1 and about 20 mbar.

When actuating the inhalation device, as the means for storing potential energy alters its state from a locked state to an unlocked state, energy is released which effects the cylinder to perform its propulsive longitudinal movement, significant pressure is built up in the pumping chamber. This generates a marked pressure difference (due to a high pressure in the pumping chamber and a substantially lower pressure in the fluid reservoir) which exceeds the threshold value of the pressure difference, so that the check valve opens and allows the pressure chamber to become filled with liquid from the reservoir.

A valve type that may be designed to operate with such a threshold pressure difference is a ball valve pre-loaded with a spring. The spring pushes the ball into its seat, and only if the pressure acting against the spring force exceeds the latter, the ball valve opens. Other valve types which—depending on their construction—may operate with such a threshold pressure difference are duckbill valves or flap valves.

The advantage of such a valve operating with a threshold pressure difference is that the reservoir can be kept closed until active use is being made of the inhalation device, thus reducing unwanted splashing of reservoir liquid during device transport, or evaporation during long-term storage of the device.

As already mentioned above, the pumping unit comprises a riser pipe having an upstream end and a downstream end, wherein the riser pipe is adapted to function as a piston in the pumping unit. Furthermore, the pumping unit of the present inhalation device comprises a holder for holding the riser pipe located downstream of the riser pipe and being firmly affixed to the user-facing side of the housing such as to be immobile relative to the housing. According to the invention, the riser pipe and the holder are in the form of a one-piece unit or, in other words, in the form of an integral one-piece unit as also described above to result in a single structure comprising the riser pipe and the holder such that the riser pipe and the holder cannot be removed or detached from each other in a non-destructive way, or, in other words, the riser pipe and the holder are provided in the form in which there is no interphase between the riser pipe and the corresponding holder.

In some embodiments, the holder and the riser pipe may comprise an injection-moldable polymeric material or, in other words, are made of a material comprising an injection-moldable polymeric material. The term “injection-moldable polymeric material” as used herein may be generally understood as any plastic polymer material that becomes pliable or moldable at a certain (elevated) temperature and that solidifies upon cooling. In some embodiments, the holder and the riser pipe comprise or are made of a material comprising at least one thermoplastic injection-moldable polymeric material. In some embodiments, the one-piece unit comprising the holder and the riser pipe may comprise different injection-moldable polymeric materials as described further below. In some embodiments, however, the holder and the riser pipe may comprise the same injection-moldable polymeric material.

In particular embodiments, the riser pipe and the holder in the form of a one-piece unit may be prepared by injection molding of at least one thermoplastic polymeric material, for example by one-step injection-molding of a selected thermoplastic polymeric material.

In particular embodiments, the one-piece unit comprising the riser pipe and the holder may comprise a thermoplastic polymeric material that may be selected from the group consisting of polyoxymethylene (POM; CAS Registry number 9002-81-7), polyether ether ketone (PEEK; CAS Registry number 29658-26-2), and poly(p-phenylene oxide (PPO) (also known as poly-p-phenylene ether (PPE); CAS Registry number 25134-01-4). In yet further particular embodiments, the injection-moldable polymeric material as comprised by the one-piece unit comprising the riser pipe and the holder of the present inhalation device and pumping unit is polyether ether ketone (PEEK).

In further embodiments, the injection-moldable polymeric material as comprised by the one-piece unit comprising the riser pipe and the holder of the present inhalation device may be a mixture or blend comprising at least one of PEEK, PPO/PPE and POM and at least one further injection-moldable polymeric material selected from the group consisting of acrylonitrile butadiene styrenes (ABS), acrylonitrile styrene acrylate (ASA), styrene acrylonitrile copolymer (SAN), polyacrylic acids, (such as poly(methyl methacrylate (PMMA)), acrylonitrile butadiene styrene (ABS), polyamides, polylactic acids (polylactide, PLA), polybenzimidazoles (PBI), polycarbonates (PC), polyether sulfone (PES), polyoxymethylenes (POM), polyether ether ketone (PEEK), polyetherimide (PEI), polyethylenes (PE), such as ultra-high-molecular-weight polyethylene (UHMWPE), high-density polyethylene (HDPE), medium-density polyethylene (MDPE), low-density polyethylene (LDPE), polyphenylene oxide (PPO), polyphenylene sulfide (PPS), polypropylene (PP), polystyrene (PS), polyvinyl chloride (PVC), polyvinylidene fluoride (PVDF) and polytetrafluorethylene (PTFE).

In further particular embodiments, the injection-moldable polymeric material may be a mixture or blend comprising polyether ether ketone (PEEK) and at least one of a polybenzimidazole (PBI, e.g. poly[2,2′-(m-phenylen)-5,5′-bisbenzimidazole]; CAS number 32075-68-6), a polylactic acid (polylactide, PLA) and polyether sulfone (PESU/PES; CAS Registry number 25608-63-3). In yet further particular embodiments, the injection moldable material may be a mixture or blend comprising poly(p-phenylene oxide (PPO/PPE) and a polystyrene (resulting in a material commercially available as Noryl™). In yet further particular embodiments, the injection moldable material may be a mixture or blend comprising a polyoxymethylene (POM) and polytetrafluorethylene (PTFE).

In some embodiments, the one-piece unit comprising the riser pipe and the holder as comprised by the present inhalation device may essentially consist of the injection-moldable material as described above, wherein the term “essentially consists” as used in this regard herein means that the piston consists of at least 85 wt.-% (compared to the total weight of the piston) or of at least 90 wt.-% or of at least 95 wt.-% or of at least 97 wt.-% or of at least 98 wt.-% or of at least 99 wt.-% of the chosen thermoplastic polymeric material.

In further particular embodiments, the riser pipe comprises a fluid channel which may be formed by an injection-moldable material as described above.

In further embodiments, the one-piece unit comprising the riser pipe and the holder may comprise a composite material comprising an injection-moldable material. In these embodiments, the composite material, in addition to the injection-moldable material, may comprise a non-injection-moldable material. In some embodiments, the non-injection-moldable material as comprised by the composite material may be at least one of the materials selected from glass, metals, ceramics, 2 DPA-1 (for example in the form of fibers, beads, granules or other suitable structures) or other solid materials, for example carbon fiber. In some embodiments, the non-injection-moldable material may be introduced directly into the chosen injection-moldable material and the resulting mixture may be injection-molded, for example in the case of glass or carbon fiber. In alternative embodiments, the non-injection-moldable-material may be introduced into the mold and over-molded by the injection-moldable material as also known to those of skill in the art as “insert-molding”.

In further embodiments, only a part of the one-piece unit comprising the riser pipe and the holder may comprise or essentially consist of a composite material as described above, while other portions of the one-piece unit do not comprise a non-injection-moldable material. For example, the riser pipe may not comprise a non-injection-moldable material and essentially consist of a thermoplastic injection-moldable material as described above while the holder may comprise one or more non-injection-moldable materials as described above. This may be advantageous especially in embodiments in which the riser pipe and the holder have different dimensions and, accordingly, different requirements, for example, with regard to the mechanical stability.

In exemplary embodiments, the riser pipe may be a thin elongate structure having an inner fluid channel and a circular cross-sectional diameter whereas the corresponding holder may be a mechanically more robust structure supporting the riser pipe. In these embodiments, for example, the holder may have larger dimensions, especially a larger diameter and may comprise a mechanically more robust material such as a composite material while the riser pipe may comprise or essentially consist of a thermoplastic injection-moldable material such as PEEK, POM or PPO/PPE. Accordingly, in some embodiments, the riser pipe as comprised by the one-piece unit may essentially consist of the injection-moldable material such as PEEK, POM or PPO/PPE while the holder may comprise the same injection-moldable material and, in addition to this, further injection-moldable or non-injection-moldable materials, for example in the form of a composite material as described above. The holder or, in other words, the holder-segment of the one-piece unit comprising the holder and the riser pipe (or, in other words, a holder-segment and a riser pipe-segment) may have all suitable sizes and cross-sectional shapes, for example, may be in the form of a cylinder with a round, oval, square, star-shaped or other cross-sectional shape as long as such shapes and sizes are suitable to affix the one-piece unit comprising the riser pipe and the holder to the housing in a mechanically stable way.

As a further advantage, in these embodiments the holder may further comprise additional functional elements, for example a filter structure located downstream of the riser pipe (and being fluidically connected to the riser pipe) or even a cascade of a plurality of (consecutive) filters. This also allows for an advantageous design of the riser pipe and corresponding holder in which no separate additional holding structures for such filter structures are necessary.

As already mentioned above, the riser pipe comprises a fluid channel which may be formed of an injection-moldable material as described above. In some embodiments, the fluid channel fluidically connects the upstream end of the riser pipe with the downstream end of the riser pipe for conveying the medically active fluid. In some embodiments, the fluid channel may have a diameter, preferably a constant diameter, of up to about 5 mm, or of up to about 4 mm, or of up to about 3 mm, or of up to about 2 mm or selected within the range of from about 0.05 mm to about 5 mm, or selected within the range of from about 0.1 mm to about 4 mm, or to about 3 mm, or to about 2 mm, or selected within the range of from about 0.1 mm to about 1 mm, or selected within the range of from about 0.2 mm to about 0.8 mm; or selected within the range of from about 0.03 mm to about 0.7 mm, or selected within the range of from about 0.4 mm to about 0.6 mm, for example 0.5 mm. In further embodiments, the riser pipe may have a (outer) diameter (perpendicular to the main axis connecting the upstream end of the riser pipe with the downstream end of the riser pipe) selected within a range of from about 1 mm to about 10 mm, or to about 7.5 mm or to about 5 mm, or to about 3 mm, or to about 2 mm selected within the range of from about 1.5 mm to about to about 2 mm, for example 1.5 mm or 1.8 mm.

In yet further embodiments, the riser pipe may have a length selected within the range of from about 10 mm to about 30 mm, from about 15 mm to about 25 mm or to about 20 mm, for example about 17 mm.

Furthermore, the riser pipe may comprise a coating applied to the outer surface of the piston, for example a coating polytetrafluorethylene (PTFE). In some embodiments, however, the piston does not comprise a surface coating.

The hollow cylinder as comprised by the pumping unit of the present inhalation device, in some embodiments, may comprise or essentially consist of a metal or a polymeric material selected from the group consisting of acrylonitrile butadiene styrenes (ABS), acrylonitrile styrene acrylate (ASA), styrene acrylonitrile copolymer (SAN), acrylonitrile butadiene styrene (ABS), polyamides, polylactic acids (polylactide, PLA), polybenzimidazoles (PBI), polycarbonates (PC), polyether sulfone (PES), polyoxymethylenes (POM), polyether ether ketone (PEEK) and polyphenylene oxide (PPO).

In particular embodiments, the pump cylinder comprises or essentially consists of a thermoplastic polymeric material selected from polyoxymethylene (POM; CAS Registry number 9002-81-7), polyether ether ketone (PEEK; CAS Registry number 29658-26-2), and poly(p-phenylene oxide (PPO) (also known as poly-p-phenylene ether (PPE); CAS Registry number 25134-01-4). In yet further embodiments, the pump cylinder comprises or essentially consists of PEEK, either in the form of a composite material or in the form of a non-composite material, i.e. without comprising any non-injection-moldable materials.

In further embodiments, the pump cylinder may comprise the thermoplastic polymeric material in the form of a composite material as described in detail above in connection with the one-piece unit comprising the riser pipe and corresponding holder.

It has been surprisingly found that the riser pipe and the holder of the pumping unit of the present inhalation device may be prepared as a one-piece unit, preferably from an injection-moldable material, despite of the considerable physical and mechanical stress acting on the riser pipe and supporting holder during operation of the pumping unit resulting from the generation of fluid pressures as high as about 100 bar or even higher. Furthermore, it has been surprisingly found that a riser pipe comprising or even essentially consisting of an injection-moldable material may meet the high physical and mechanical requirements that should be met by a riser pipe acting as a piston in a medical high-pressure pumping unit such as mechanical and chemical stability, surface properties such as roughness, dimension accuracy, temperature and pressure intolerance, chemical neutrality and resistance as well as hardness. This is especially surprising in view of the fact that the riser pipe as comprised by the one-piece unit also comprising the holder is hollow and, accordingly comprises a fluid channel extending along the main axis of the riser pipe. Despite of the high physical and mechanical requirements combined with the fine structures of the riser pipe as, for example, the inner fluid channel, it was surprisingly found that the riser pipe may be manufactured by injection molding of a material comprising an injection-moldable polymeric material in the form of a one-piece unit also comprising the holder. This, however, may be considered as a significant advantage when compared to current corresponding riser pipe structures manufactured from a metal such as stainless steel which have to be reliably affixed to the supporting holding structure in a separate manufacturing step. Furthermore, the present design of the riser pipe and corresponding holder in the form of a one-piece structure allows for the closest possible positioning of the riser pipe relative to all functional structures located downstream of the riser pipe, such as an optional prefilter and filter and/or the nozzle which allows to avoid any hollow or dead spaces before these structures.

With respect to surface properties of the riser pipe and holder (e.g., the riser pipe segment and the holder segment of the holder), the surface may at least partially or wholly have an average surface roughness (RA) between 0.010 to 0.060 μm, between 0.010 to 0.030 μm, preferably between 0.012 to 0.025 μm or between 0.025 to 0.050 μm. In an aspect of the disclosure where the riser pipe and holder comprises a composite material, the surface may have a molding surface finish in accordance with the standard SPI A2. In particular, it is advantageous to provide the riser pipe and holder with these surface properties at interfaces with other structures. For example, at an interface between the riser pipe and holder to the nozzle, discussed above, these surface properties ensure advantageous interaction between the surface and, for example, a seal, such as an O-ring, that may ensure a proper seal between the nozzle and the riser pipe and holder.

The inventors found further advantages of the use of injection-moldable materials. It was surprisingly found that a piston made of injection moldable material is suitable for use in high-pressure medical pumping device, such as an inhalation device according to the present invention.

As such, in one aspect the invention relates to a high-pressure medical pumping device for providing discrete portions of a medical fluid in pressurized form, wherein the pumping device comprises
a fluid inlet;
a fluid outlet; and
a pumping unit fluidically connecting the fluid inlet with the fluid outlet and being adapted to generate the discrete portions of the medical fluid in pressurized form and to convey the discrete portions of the medically active fluid (in a downstream direction) from the fluid inlet to the fluid outlet;
wherein the pumping unit comprises
a pump cylinder comprising a pump chamber located within the pump cylinder, the pump chamber comprising a fluid opening; and
a piston comprising a first end and a second end located opposite of the first end, wherein the piston is at least partly located within the pump chamber of the pump cylinder such that at least the first end of the piston is located within the pump chamber, and wherein the piston and the pump cylinder are arranged for back-and-forth stroke movement of at least one of the piston and the pump chamber relative to each other,
wherein the piston comprises a fluid channel fluidically connecting the first end of the piston with the second end of the piston and being adapted to convey the medical fluid from the first or second end of the piston to the corresponding opposite end of the piston; and
wherein the pumping unit is adapted to provide the discrete portions of the medical fluid in pressurized form at a pressure of at least 100 bar; and
wherein the piston comprises an injection-moldable polymeric material.

In a particular embodiment, the invention relates to a hand-held inhalation device for delivering a medically active fluid comprising a high-pressure medical-pumping device as defined above.

The material of said piston may be as defined above.

In further embodiments, the present inhalation device may further comprise an outlet valve inside the riser pipe, or at an end of the riser pipe, for avoiding a return flow of liquid or air from the riser pipe into the hollow cylinder. In many cases, the use of such outlet valve will prove to be advantageous. Typically, the downstream end of the riser pipe is located close to the nozzle. The nozzle is in fluidic communication with the outside air. After emitting, in aerosolized form, the amount of liquid which is delivered from the pumping unit through the nozzle, driven by the propulsive longitudinal movement of the cylinder, the pumping chamber must be refilled. For this purpose, it slides back on the riser pipe into its previous upstream position (i.e. performs a repulsive longitudinal movement), so that the interior volume of the pumping chamber increases. Along with this, a negative pressure (sometimes also referred to as “underpressure”) is generated inside the pumping chamber which causes liquid to be sucked into the pumping chamber from the fluid reservoir which is located upstream of the pumping chamber. However, such negative pressure may also propagate downstream through the riser pipe up to the outside of the nozzle and could lead to air being sucked into the device through the nozzle, or nozzle openings, respectively. This problem can be avoided by providing an outlet valve, also referred to as outlet check valve, which opens towards the nozzle openings and blocks in the opposite direction.

Optionally, the outlet valve may be of a type that blocks below (and opens above) a threshold pressure difference as described in the context of the inlet valve above. If a ball valve with a spring is used, the spring force must be directed against the pumping chamber such that when the difference between the interior pressure of the pumping chamber and the ambient pressure exceeds the threshold pressure difference value, the outlet valve opens. The advantages of such a valve correspond to the respective aforementioned advantages.

As mentioned, the outlet valve may be positioned within the riser pipe. Alternatively, the inhalation device may comprise an outlet valve which is not integrated within the riser pipe, but positioned at or near one of the ends of the riser pipe, in particular at or near its downstream end, e.g. in the holder located downstream of the riser pipe for holding the riser pipe as described above. This embodiment may be advantageous in certain cases, e.g. if there is a need for a riser pipe with a particularly small diameter which makes the integration of a valve difficult. By accommodating the outlet valve downstream of the riser pipe, a valve with a relatively large diameter may be used, thus simplifying the requirements for the valve design.

In alternative embodiments, the outlet valve may be absent. These embodiments may be feasible as the fluid channels of an impingement-type nozzle may have relatively small cross sections, resulting in only minor or very slow back flow at the given pressure conditions during the priming of the device. If the amount of backflow is considered acceptable in view of a particular product application, the inhaler design may be simplified by avoiding the outlet valve.

In any case, whether the inhalation device is designed with or without an outlet valve, all other options and preferences described with respect to other device features are applicable to both of these alternative embodiments.

In further embodiments, the present inhalation device may comprise a fluid reservoir which is firmly attached to the hollow cylinder such as to be moveable together with the hollow cylinder inside the housing. This means that in each ejection phase of the pumping cycle, the fluid reservoir moves together with the hollow cylinder from an initial (“upstream”) position, in which the pumping chamber has its maximum interior volume, towards an end (“downstream”) position, in which the volume of the pumping chamber is minimal; and during the subsequent “priming” step, the fluid reservoir returns together with the hollow cylinder to their initial (“upstream”) position.

As used herein, the expression “firmly attached” includes both permanent and non-permanent (i.e. releasable) forms of attachment. Moreover, it includes direct and indirect (i.e. via one or more connecting parts) types of attachment. At the same time, as mentioned above, “firmly attached” means that the respective parts are fixed to each other in such a way as to substantially prevent their movement relative to each other. In other words, two parts that are firmly attached to each other may only be movable together, and with respect to each other, they are non-movable or immobile.

One of the advantages of this embodiment wherein the fluid reservoir is firmly attached to the hollow cylinder is that it provides the smallest possible dead volume between the reservoir and the pumping chamber.

According to an alternative embodiment, the fluid reservoir may be fluidically connected to the hollow cylinder by means of a flexible tubular element, and firmly attached to the housing. According to this embodiment, the reservoir is not firmly attached to the hollow cylinder and does not move along with it when the cylinder performs its longitudinal movements. Instead, it is firmly, but optionally detachably, directly or indirectly, attached to the housing or to a part of the housing. One advantage of this embodiment is that the energy which is abruptly released upon unlocking the means for storing potential energy solely acts on the hollow cylinder and not on the fluid reservoir. This may be particularly advantageous in cases in which the fluid reservoir in its initial (fully filled state) at the beginning of its usage has a relatively large mass which decreases overuse. A higher acceleration of the hollow cylinder would translate into a higher pressure in the pumping chamber.

For the avoidance of doubt, all other options and preferences described herein-above and below with respect to other device features are applicable to both of these alternatives, i.e. regardless of whether the fluid reservoir is firmly attached to the hollow cylinder or not.

In some embodiments, the fluid reservoir is designed to be collapsible, such as by means of a flexible or elastic wall. The effect of such design is that upon repeated use of the device which involves progressive emptying of the reservoir, the flexible or elastic wall buckles or folds such as to reduce the internal volume of the reservoir, so that the negative pressure which is necessary for extraction of a certain amount of liquid is not required to increase substantially over the period of use. In particular, the reservoir may be designed as a collapsible bag. The advantage of a collapsible bag is that the pressure inside the reservoir is almost independent of the filling level, and the influence of thermal expansion is almost negligible. Also, the construction of such a reservoir type is rather simple and already well established.

A similar effect can be achieved with a rigid container which has a moveable bottom (or wall) by means of which the interior volume of the reservoir can also be successively reduced.

In a second aspect, the present invention provides for a one-piece unit comprising a riser pipe and a holder adapted to be comprised by the inhalation device according to the first aspect of the invention. The one-piece unit according to this aspect of the invention is adapted to be comprised by the inhalation device according to the first aspect of the invention, more specifically adapted to be comprised (as a piston) by the pumping unit of the inhalation device according to the first aspect of the invention.

For the avoidance of doubt, it should be noted that all embodiments, features, definitions, explanations and combinations thereof as disclosed in connection with the pumping device of the first aspect of the invention also apply to this second aspect of the invention as well as all further aspects of the invention although not being repeated in connection with the present and all further aspects of the invention.

In a third aspect, the present invention provides for a method for the manufacture of a one-piece unit comprising a riser pipe and a corresponding holder according to the second aspect of the invention, wherein the method comprises injection-molding of an injection-moldable material. In this context, it should be noted that general techniques of injection-molding of injection moldable polymeric materials, optionally in the form of composite materials are known to those of skill in the art and well documented in the corresponding standard literature, see for example Plastics Technology Handbook, M. Chanda, CRC Press, November 2017, ISBN 9781498786218.

In a fourth aspect, the invention provides a method for the generation of a nebulized aerosol of a medically active liquid. The method may involve the use of the inhalation device of the first aspect of the invention as described herein-above, in any of its embodiments. Therefore, all options and preferences described above with respect to the features of the inhalation device are also applicable to the method provided according to the invention.

In particular, the method comprises the steps of:
(a) providing a hand-held inhalation device according to the first aspect of the invention, wherein the fluid reservoir of the inhalation device comprises a medically active liquid, and wherein the means for storing potential energy is in an unlocked state;
(b) priming said inhalation device by causing the means for storing potential energy to alter its state from an unlocked state to a locked state, whereby the hollow cylinder performs a repulsive longitudinal movement on the riser pipe towards the upstream end of the pumping unit such as to allow medically active liquid to flow from the fluid reservoir into the hollow cylinder; and subsequently
(c) actuating said inhalation device by causing the means for storing potential energy to become unlocked, whereby a propulsive longitudinal movement of the cylinder towards the downstream end of the pumping unit is effected and medically active liquid is ejected in a downstream direction from the hollow cylinder through the nozzle.

According to the invention, during the movement of the hollow cylinder relative to the housing and to the nozzle, the riser pipe stays immobile, while the cylinder which provides the pumping chamber alters its relative position. In other words, the pumping chamber is moveably supported within the housing, whereas riser pipe and nozzle are firmly and immovably, or stationary, attached to the housing and/or nozzle via the holder.

In that it relies on the use of a particularly advantageous inhalation device, the method provides an improvement to the methods known from the art, wherein the component corresponding to the riser pipe, namely the hollow piston, is moveable with respect to housing and nozzle, and the riser pipe is stationary.

According to further embodiments, the described method may be carried out by means of an inhalation device that provides one, several, or all of the aforementioned features, such as: a reservoir, being firmly attached to the hollow cylinder or being firmly attached to the housing, and optionally being provided as a collapsible bag; an inlet check valve, optionally operable by using a threshold pressure difference; a means for the storage of potential energy such as e.g. a spring; a locking device; and an outlet valve, being e.g. arranged inside the riser pipe or between riser pipe and nozzle. For explanations regarding the respective operation of said features, reference is made to the sections of the description that relate to the respective device features.

According to a further specific and advantageous embodiment, the invention provides a method for the generation of an aerosol of a medically active liquid by means of an inhalation device, the device comprising a housing, a pumping device with a pumping chamber, a riser pipe with a holder being a one-piece unit, and a nozzle, by performing the steps of
moving an interior end of the riser pipe into the interior volume of the pumping chamber such that the latter is reduced and a pressure of a liquid contained within this volume is increased; and
ejecting said liquid through an exterior end of said riser pipe and the nozzle such that a spray is formed;
   characterized in that during said motion, with regard to housing and/or nozzle, said riser pipe stays immobile, while said pumping chamber alters its relative position.

As has been shown above, the invention solves the drawbacks of the known art, especially regarding cost and number of process step during manufacture. It also allows for the nebulization and delivery of aerosols from liquids of higher viscosities in a short time, and with high reproducibility.

The following is a list of numbered embodiments E1 to E32 comprised by the present invention:

E1. A hand-held inhalation device for delivering a medically active fluid (F) in aerosolized form for inhalation therapy, comprising
(a) a housing (1) having a user-facing side;
(b) a nozzle (6) for generating the nebulized aerosol by collision of at least two liquid jets, the nozzle (6) being firmly affixed to the user-facing side of the housing (1) such as to be immobile relative to the housing (1);
(c) a fluid reservoir (2) arranged within the housing (1) for holding the medically active fluid; and
(d) a pumping unit arranged within the housing (1), the pumping unit having
an upstream end that is fluidically connected to the fluid reservoir (2); and
a downstream end that is fluidically connected to the nozzle (6); wherein the pumping unit is adapted for pumping the medically active fluid (F) from the fluid reservoir (2) to the nozzle (6);

wherein the pumping unit further comprises
(i) a riser pipe (5) having an upstream end and a downstream end, wherein the riser pipe (5) is adapted to function as a piston in the pumping unit;
(ii) a holder (10) located downstream of the riser pipe being firmly affixed to the user-facing side of the housing (1) such as to be immobile relative to the housing (1) for holding the riser pipe;
(iii) a hollow cylinder (9) located upstream of the riser pipe (5), wherein the upstream end of the riser pipe (5) is inserted in the cylinder (9) such that the cylinder (9) is longitudinally movable on the riser pipe (5); and
(iv) a lockable means for storing potential energy (7) when locked and for releasing the stored energy when unlocked, the means (7) being arranged outside of, and mechanically coupled to, the cylinder (9) such that unlocking the means (7) results in a propulsive longitudinal movement of the cylinder (9) towards the downstream end of the pumping unit;
wherein the holder (10) and the riser pipe (5) are a one-piece unit.

E2. The inhalation device according to claim 1, wherein the holder (10) and the riser pipe (5) comprise an injection-moldable polymeric material.

E3. The inhalation device according to embodiment E1 or E2, wherein the holder (10) and the riser pipe (5) are made of a material comprising at least one thermoplastic injection-moldable polymeric material.

E4. The inhalation device according to any one of the preceding embodiments, wherein the holder (10) and the riser pipe (5) comprise the same injection-moldable polymeric material.

E5. The inhalation device according to any one of the preceding embodiments, wherein the riser pipe (5) and the holder (10) in the form of a one-piece unit are prepared by injection molding of at least one thermoplastic polymeric material.

E6. The inhalation device according to any one of the preceding embodiments, wherein the injection-moldable polymeric material is selected from the group consisting of polyoxymethylene (POM), polyether ether ketone (PEEK), and polyphenylene oxide (PPO/PPE).

E7. The inhalation device according to any one of the preceding embodiments, wherein the injection-moldable polymeric material is a mixture or blend comprising at least one injection-moldable material selected from the group consisting of PEEK, PPO/PPE and POM and at least one further injection-moldable polymeric material selected from the group consisting of acrylonitrile butadiene styrenes (ABS), acrylonitrile styrene acrylate (ASA), styrene acrylonitrile copolymer (SAN), polyacrylic acids, (such as poly(methyl methacrylate (PMMA)), acrylonitrile butadiene styrene (ABS), polyamides, polylactic acids (polylactide, PLA), polybenzimidazoles (PBI), polycarbonates (PC), polyether sulfone (PES), polyoxymethylenes (POM), polyether ether ketone (PEEK), polyetherimide (PEI), polyethylenes (PE), such as ultra-high-molecular-weight polyethylene (UHMWPE), high-density polyethylene (HDPE), medium-density polyethylene (MDPE), low-density polyethylene (LDPE), polyphenylene oxide (PPO), polyphenylene sulfide (PPS), polypropylene (PP), polystyrene (PS), polyvinyl chloride (PVC), polyvinylidene fluoride (PVDF) and polytetrafluorethylene (PTFE).

E8. The inhalation device according to any one of the preceding embodiments, wherein the at least one injection-moldable material is polyether ether ketone (PEEK).

E9. The inhalation device according to any one of the preceding embodiments, wherein the piston comprises a composite material comprising an injection-moldable material.

E10. The inhalation device according to embodiment E9, wherein the composite material, in addition to the injection moldable material, comprises a non-injection-moldable material.

E11. The inhalation device according to embodiment E9 or E10, wherein the composite material comprises at least one non-injection-moldable material selected from glass, metals, ceramics, 2 DPA-1 (for example in the form of fibers, beads, granules or other suitable structures) or other solid materials, for example carbon fiber.

E12. The inhalation device according to any one of the preceding embodiments, wherein the riser pipe essentially consists of the injection-moldable material.

E13. The inhalation device according to any one of the preceding claims, wherein the fluid channel (11) of the riser pipe is formed by an injection-moldable material.

E14. The inhalation device according to any one of the preceding embodiments, wherein the hollow cylinder comprises a metal or a polymeric material acrylonitrile butadiene styrenes (ABS), acrylonitrile styrene acrylate (ASA), styrene acrylonitrile copolymer (SAN), acrylonitrile butadiene styrene (ABS), polyamides, polylactic acids (polylactide, PLA), polybenzimidazoles (PBI), polycarbonates (PC), polyether sulfone (PES), polyoxymethylenes (POM), polyether ether ketone (PEEK) and polyphenylene oxide (PPO)

E15. The inhalation device according to any one of the preceding embodiments, wherein the riser pipe comprises a fluid channel fluidically connecting the upstream end of the riser pipe with the downstream end of the riser pipe for conveying the medically active fluid.

E16. The inhalation device according to embodiment E15, wherein the fluid channel has a diameter selected within the range of from about 0.1 mm to about 1 mm.

E17. The inhalation device according to any one of the preceding embodiments, wherein the riser pipe has a diameter (perpendicular to the main axis connecting the upstream end of the riser pipe with the downstream end of the riser pipe) selected within a range of from about 1 mm to about 3 mm.

E18. The inhalation device according to any one of the preceding embodiments, wherein the holder further comprises a filter structure located downstream of the riser pipe (and being fluidically connected to the riser pipe).

E19. The inhalation device according to embodiment E18, wherein the filter structure comprises a cascade of a plurality of (consecutive) filters.

E20. The inhalation device according to any one of the preceding embodiments, wherein the nozzle comprises an impingement-type nozzle.

E21. The inhalation device according to any one of the preceding embodiments, wherein the pumping unit further comprises a lock for locking the lockable means.

E22. The inhalation device according to any one of the preceding embodiments, wherein the pumping unit is a high-pressure pumping unit adapted to expel fluid (F) at a pressure of at least 50 bar.

E23. The inhalation device according to any one of the preceding embodiments, wherein the means for storing potential energy (7) is a spring having a load of at least 10 N in a deflected state.

E24. The inhalation device according to any one of the preceding embodiments, being adapted to deliver the nebulized medically active aerosol discontinuously in the form of discrete units, wherein one unit is delivered per pumping cycle.

E25. The inhalation device according to embodiment E24, wherein a unit of the aerosol contains from 2 μL to 150 μL of liquid phase.

E26. The inhalation device according to any one of the preceding embodiments, wherein the pumping unit further comprises an inlet valve (4) positioned downstream or at the downstream end of the hollow cylinder (9).

E27. The inhalation device according to embodiment E26, wherein the inlet valve (4) is adapted to open only when the pressure difference between an upstream and a downstream side of the inlet valve (4) is above a predefined threshold value, and wherein the predefined threshold value is in the range from 1 to 20 mbar.

E28. The inhalation device according to any one of the preceding embodiments, further comprising an outlet valve (8) inside the riser pipe (5) for avoiding a return flow of liquid (F) or air from the riser pipe (5) into the hollow cylinder (9).

E29. The inhalation device according to any one of the preceding embodiments, wherein the fluid reservoir (2) is firmly attached to the hollow cylinder (9) such as to be moveable together with the hollow cylinder (9) inside the housing (1).

E30. The inhalation device according to any one of the preceding embodiments, wherein the fluid reservoir (2) is fluidically connected to the hollow cylinder (3) by means of a flexible tubular element, and firmly attached to the housing (1).

E31. The inhalation device according to any one of the preceding embodiments, wherein the fluid reservoir (2) is designed as a collapsible bag.

E32. A method for the generation of a nebulized aerosol of a medically active liquid (F) comprising the steps of:
providing a hand-held inhalation device as defined in any one of the preceding embodiments, wherein the fluid reservoir (2) of the inhalation device comprises a medically active liquid (F), and wherein the means for storing potential energy (7) is in an unlocked state;
priming said inhalation device by causing the means for storing potential energy (7) to alter its state from an unlocked state to a locked state, whereby the hollow cylinder (9) performs a repulsive longitudinal movement on the riser pipe (5) towards the upstream end of the pumping unit such as to allow medically active liquid (F) to flow from the fluid reservoir (2) into the hollow cylinder (9); and subsequently
actuating said inhalation device by causing the means for storing potential energy (7) to become unlocked, whereby a propulsive longitudinal movement of the cylinder (9) towards the downstream end of the pumping unit is effected and medically active liquid (F) is ejected in a downstream direction from the hollow cylinder (9) through the nozzle (6).

In a particular embodiment the invention relates the following further Embodiments directed at a high-pressure medical pumping device comprising a piston made of an injection moldable material:

A1. A high-pressure medical pumping device for providing discrete portions of a medical fluid in pressurized form, wherein the pumping device comprises
a fluid inlet;
a fluid outlet; and
a pumping unit fluidically connecting the fluid inlet with the fluid outlet and being adapted to generate the discrete portions of the medical fluid in pressurized form and to convey the discrete portions of the medically active fluid (in a downstream direction) from the fluid inlet to the fluid outlet;
wherein the pumping unit comprises
a pump cylinder comprising a pump chamber located within the pump cylinder, the pump chamber comprising a fluid opening; and
a piston comprising a first end and a second end located opposite of the first end, wherein the piston is at least partly located within the pump chamber of the pump cylinder such that at least the first end of the piston is located within the pump chamber, and wherein the piston and the pump cylinder are arranged for back-and-forth stroke movement of at least one of the piston and the pump chamber relative to each other,
wherein the piston comprises a fluid channel fluidically connecting the first end of the piston with the second end of the piston and being adapted to convey the medical fluid from the first or second end of the piston to the corresponding opposite end of the piston; and
wherein the pumping unit is adapted to provide the discrete portions of the medical fluid in pressurized form at a pressure of at least about 100 bar; and
wherein the piston comprises an injection-moldable polymeric material.

A2. The high-pressure medical pumping device according to embodiment A1, wherein the medical pumping unit is adapted to provide the discrete portions of the medical fluid in pressurized form at a pressure of at least about 150 bar, or of at least about 175 bar or of at least about 200 bar.

A3. The high-pressure medical pumping device according to embodiment A1 or A2, wherein the medical pumping device comprises a check valve fluidically connecting the fluid inlet with the pumping unit (and being located between the fluid inlet and the pumping unit).

A4. The high-pressure medical pumping device according to any one of the preceding embodiments, wherein the pumping device is adapted to be comprised by a mobile inhalation device, preferably by a hand-held mobile inhalation device.

A5 The high-pressure medical pumping device according to any one of the preceding embodiments, wherein the pumping device is adapted to be comprised by an inhalation device for the administration of the medical fluid in aerosolized form for inhalation.

A6. The high-pressure medical pumping device according to any one of the preceding embodiments, wherein the liquid inlet is fluidically connected to a reservoir for holding the medical fluid, for example a reservoir in the form of a dimensionally stable or flexible container.

A7. The high-pressure medical pumping device according to any one of the preceding embodiments, wherein the fluid outlet is fluidically connected to a nozzle for discharging and optionally aerosolizing the conveyed medical fluid in pressurized form, for example an impingement-type nozzle for aerosolizing the medical fluid by collision of at least two liquid jets of the medical fluid.

A8. The high-pressure medical pumping device according to any one of the preceding embodiments, wherein the fluid opening of the pump cylinder is fluidically connected to the fluid inlet of the pumping device (and optionally to the reservoir) and located downstream of the fluid inlet of the pumping device and wherein the second end of the piston is fluidically connected to the fluid outlet of the pumping device.

A9. The high-pressure medical pumping device according to embodiment A8, wherein the pumping unit comprises a check valve and wherein the check valve is comprised by the pump cylinder and/or is positioned adjacent to the fluid inlet of the pump cylinder.

A10. The high-pressure medical pumping device according to embodiment A8 or A9, wherein the piston is arranged in a fixed position within the pumping unit such that the piston is immobile during back-and-forth stroke movement of the pump chamber.

A11. The high-pressure medical pumping device according to any one of embodiments A1 to A8, wherein fluid opening of the pump cylinder is fluidically connected to the fluid outlet of the pumping device and located upstream of the fluid outlet of the pumping device and wherein the second end of the piston is connected to the fluid inlet of the pumping device.

A12. The high-pressure medical pumping device according to embodiment A11, wherein the pumping unit comprises a check valve comprised by the piston, and optionally positioned at the first end of the piston.

A13. The high-pressure medical pumping device according to embodiment A11 or A12, wherein the pump cylinder is arranged in a fixed position within the pumping unit such that the pump cylinder is immobile during back-and-forth stroke movement of the piston.

A14. The high-pressure medical pumping device according to any one of the preceding embodiments, wherein one discrete portion of the medical fluid in pressurized form is generated by one stroke move of at least one of the piston and the pump cylinder.

A15. The high-pressure medical pumping device according to any one of the preceding embodiments, wherein the injection-moldable material is a thermoplastic polymeric material.

A16. The high-pressure medical pumping device according to any one of the preceding embodiments, wherein the injection-moldable polymeric material is selected from the group consisting of polyoxymethylene (POM), polyether ether ketone (PEEK), and polyphenylene oxide (PPO/PPE).

A17. The high-pressure medical pumping device according to any one of the preceding embodiments, wherein the injection-moldable polymeric material is a mixture or blend comprising at least one injection-moldable material selected from the group consisting of PEEK, PPO/PPE and POM and at least one further injection-moldable polymeric material selected from the group consisting of polyacrylic acids, (such as poly(methyl methacrylate (PMMA)), acrylonitrile butadiene styrene (ABS), polyamides, polylactic acid (polylactide, PLA), polybenzimidazole PBI), polyether sulfone (PES), polyoxymethylene (POM), polyether ether ketone (PEEK), polyetherimide (PEI), polyethylene (PE, such as ultra-high-molecular-weight polyethylene (UHMWPE), high-density polyethylene (HDPE), medium-density polyethylene (MDPE), low-density polyethylene (LDPE)), polyphenylene oxide (PPO), polyphenylene sulfide (PPS), polypropylene (PP), polystyrene (PS), polyvinyl chloride (PVC), polyvinylidene fluoride PVDF) and polytetrafluorethylene (PTFE).

A18. The high-pressure medical pumping device according to any one of the preceding embodiments, wherein the at least one injection-moldable material is polyether ether ketone (PEEK).

A19. The high-pressure medical pumping device according to any one of the preceding embodiments, wherein the piston comprises a composite material comprising an injection-moldable material.

A20. The high-pressure medical pumping device according to embodiment A19, wherein the composite material, in addition to the injection moldable material, comprises a non-injection-moldable material.

A21. The high-pressure medical pumping device according to embodiment A19 or A20, wherein the composite material comprises at least one non-injection-moldable material selected from glass, metals, ceramics, 2 DPA-1 (for example in the form of fibers, beads, granules or other suitable structures) or other solid materials, for example carbon fiber.

A22. The high-pressure medical pumping device according to any one of the preceding embodiments, wherein the piston essentially consists of the injection-moldable material.

A23. The high-pressure medical pumping device according to any one of the preceding embodiments, wherein the fluid channel of the piston is formed by an injection-moldable material.

A24. The high-pressure medical pumping device according to any one of the preceding embodiments, wherein the pump cylinder as comprised by the pumping unit of the present high-pressure medical pumping device may comprise or essentially consist of a metal or a polymeric material selected from the group consisting of acrylonitrile butadiene styrenes (ABS), acrylonitrile styrene acrylate (ASA), styrene acrylonitrile copolymer (SAN), acrylonitrile butadiene styrene (ABS), polyamides, polylactic acids (polylactide, PLA), polybenzimidazoles (PBI), polycarbonates (PC), polyether sulfone (PES), polyoxymethylenes (POM), polyether ether ketone (PEEK) and polyphenylene oxide (PPO).

A25. The high-pressure medical pumping device according to any one of the preceding embodiments, wherein the fluid channel has a main axis connecting the centers of the first and second end of the piston and wherein the fluid channel has a diameter perpendicular to the main axis selected within the range of from about 0.1 mm to about 1 mm, preferably of from about 0.3 mm to about 0.7 μm.

A26. The high-pressure medical pumping device according to any one of the preceding embodiments, wherein the piston has an outer diameter (perpendicular to the main axis) selected within a range of from about 1 mm to about 10 mm, preferably of from about 1 mm to about 3 mm.

A27. The high-pressure medical pumping device according to any one of the preceding embodiments, wherein the pump chamber (as defined by the inner walls of the pump cylinder and the first end of the piston) has volume selected within the range of from about 1 μL to about 100 μL, preferably of from about 15 μL to about 30 μL.

A28. The high-pressure medical pumping device according to any one of the preceding embodiments, wherein the pumping unit comprises a drive unit adapted for driving the stroke movement of at least one of the piston and the pump cylinder.

A29. The high-pressure medical pumping device according to embodiment A28, wherein the drive unit is adapted to exert a driving force on at least one of the piston and pump cylinder in the range of from about 35 N to about 80 N.

A30. The high-pressure medical pumping device according to any one of the preceding embodiments, wherein the pumping unit comprises a lockable means for storing potential energy when locked and for releasing the stored energy when unlocked, the means being arranged outside of and being mechanically coupled to at least one of the pump cylinder and the piston such that unlocking the means results in a propulsive longitudinal movement of at least one of the pump cylinder and the piston towards the corresponding other one of the pump cylinder and the piston.

A31. The high-pressure medical pumping device according to embodiment A30, wherein the means for storing potential energy comprises a spring, such as a spring having a load of at least 35 N in a deflected state.

A32. The high-pressure medical pumping device according to embodiment A30 or A31, wherein the pumping unit further comprises a lock for locking the lockable means.

A33. The high-pressure medical pumping device according to any one of the preceding embodiments, wherein one discrete portion of the medical fluid is delivered per pumping cycle.

A34. The high-pressure medical pumping unit according to any one of the preceding embodiments, wherein one discrete portion of the medical fluid has a volume selected in the range of from 2 μL to about 150 μL.

A35. The high-pressure medical pumping device according to any one of the preceding embodiments, wherein the medical liquid is a liquid composition comprising an aqueous or alcoholic liquid vehicle and optionally at least one medically active ingredient and optionally at least one pharmaceutically acceptable excipient.

A36. An inhalation device for the administration of a medical liquid in aerosolized form comprising a high-pressure medical pumping device according to any one of embodiments A1 to A35.

A37. A piston for the pumping unit of a high-pressure medical pumping device according to any one of embodiments A1 to A36.

A38. A method for the manufacture of a piston according to embodiment A37, wherein the method comprises injection-molding of an injection-moldable material.

### DETAILED DESCRIPTION OF THE DRAWINGS

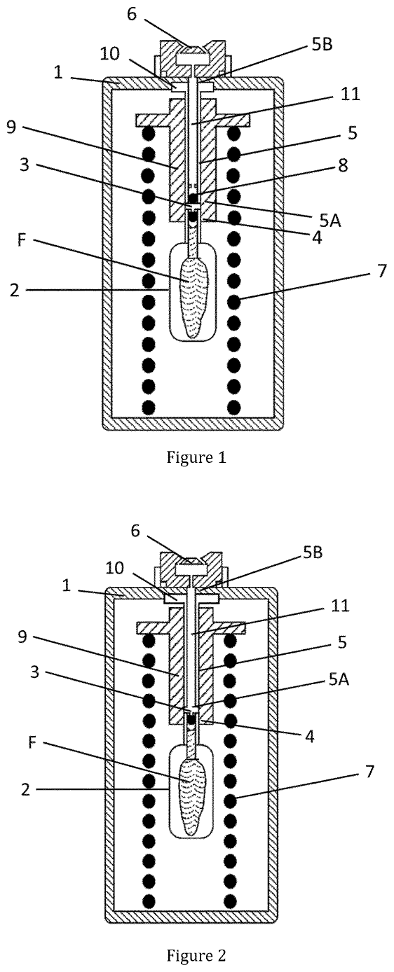

FIG. 1 depicts a schematic and not-to-scale representation of an inhalation device according to the invention. In the device as depicted, riser pipe (5) is affixed to housing (1) via holder (10) wherein riser pipe (5) and folder (1) are in the form of a one-piece unit. FIG. 1 shows the situation prior to first use.

The inhalation device comprises a housing (1), which is preferably shaped and dimensioned such that it can be held with one hand and can be operated by one finger, e.g. a thumb or index finger (not shown). A fluid reservoir (2) for the storage of a medically active liquid (F) is located inside the housing (1). The depicted reservoir (2) is designed to be collapsible so that in the course of the emptying of the reservoir by the repeated use of the device, the soft or elastic walls deform such that the negative pressure required for withdrawing liquid from the reservoir remains substantially constant over time. A similar effect could be achieved with a rigid container that has a movable bottom by means of which the interior volume of the reservoir can also successively be reduced (not shown).

Furthermore, the inhalation device comprises a pumping unit with a hollow cylinder (9) within the housing (1) which forms a pumping chamber (3) for the generation of the desired pressure which is necessary for emitting liquid (F) and nebulizing the same. The pumping unit may also comprise further components not depicted in the drawing, such as a push button, locking device, etc.

As a means for the storage of potential energy (7), a spring is provided which is coupled with one end (upwards directed, or downstream) to the cylinder (9) and which is supported at the housing (1) (lower part of the figure).

The inhalation device further comprises a riser pipe (5) with at least one reservoir-facing, or upstream, interior end (5A) which can be received in said cylinder (9). In other words, riser pipe (5) can be at least partially pushed into hollow cylinder (9), resulting in a decrease of the interior volume of pumping chamber (3). The term “interior volume” describes the volume of the space which extends from the reservoir-facing inlet of the cylinder (9) to the place where the interior end (5A) of the riser pipe (5) is located. In the depicted situation, riser pipe (5) is almost entirely contained in the cylinder (9). As a result, the interior volume of the pumping chamber (3), situated between inlet valve (4) and the interior end (5A) of riser pipe (5), is at a minimum.

Preferably, the section (or segment) of the hollow cylinder (9) which serves as or accommodates the pumping chamber (3) and which receives the riser pipe (5) exhibits a circular inner cross-section whose diameter relatively closely (e.g. except for a small gap) matches the diameter of the circular outer cross-section of the corresponding segment of the riser pipe (5). Of course, other (e.g. non-circular) cross section shapes are possible as well.

According to the depicted embodiment, inlet valve (4) is arranged between reservoir (2) and inlet of the pumping chamber (3) formed by the cylinder (9).

Furthermore, the inhalation device comprises a nozzle (6) which is connected liquid-tight to the exterior (or downstream) end (5B) of the riser pipe (5). Nozzle (6) is an impingement-type nozzle for generating the nebulized aerosol by collision of at least two liquid jets. Preferably, the cross sections of the liquid-containing channels are relatively small, typically in the region of microns.

Also depicted is an optional outlet valve (8) inside the riser pipe (5) for avoiding a backflow of liquid or air into the exterior end (5B) of the same from the outside.

Outlet valve (8) is arranged in the interior end (5A) of riser pipe (5). Liquid (F) can pass outlet valve (8) in direction of nozzle (6), but outlet valve (8) blocks any undesired backflow in the opposite direction.

As can be seen in FIG. 1, riser pipe (5) has fluid channel (11) and is designed immobile with respect to the housing (1), and firmly attached to housing (1) via holder (10). Holder (10) as shown here schematically, of course may also have all suitable sizes and cross-sectional shapes, for example, may be in the form of a cylinder with a round, oval, square, star-shaped or other cross-sectional shape. Via holder (10), riser pipe (5) (in the form of a one-piece unit with holder (10)) is also firmly attached to nozzle (6), which in turn is attached to housing (1) as well. In contrast, the hollow cylinder (9) providing the pumping chamber (3) is designed to be moveable with respect to housing (1) and nozzle (6). The benefits of this design have been explained; reference is made to the respective sections of the description above.

Referring to FIG. 2, a device similar to the one of FIG. 1 is depicted. However, the embodiment shown in FIG. 2 lacks the (optional) outlet valve (8). All other components are present, and also the function is comparable. In this embodiment, pumping chamber (3) extends from downstream of the valve (4) up to nozzle (6), which is the location where the fluidic resistance increases significantly. In an alternative embodiment having a particularly small inner diameter of riser pipe (5), pumping chamber (3) extends only from downstream of the valve (4) up to upstream interior end (5A) of riser pipe (5).

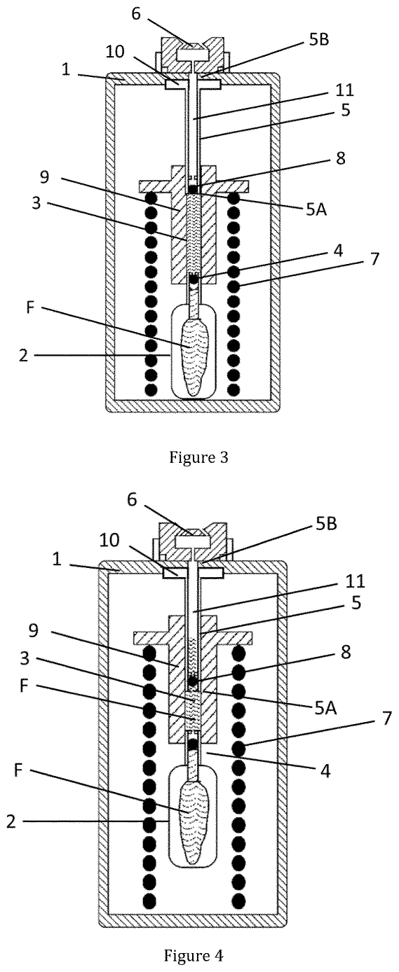

FIG. 3 shows the embodiment of FIG. 1 with a filled pumping chamber. The hollow cylinder (9) has been moved to its most upstream position, thereby loading the means for the storage of potential energy (7). Outlet valve (8) is closed due to negative pressure inside pumping chamber (3), and the inlet valve (4) is open towards the fluid reservoir (2). Increasingly collapsing walls of reservoir (2) allow the internal pressure in the reservoir (2) to remain nearly constant, while the pressure inside the pumping chamber (3) drops because of the propulsive longitudinal motion of the hollow cylinder (9), thus increasing the volume of pumping chamber (3). As a result, the pumping chamber (3) has been filled with liquid (F) from the reservoir (2).

In FIG. 4, the situation after the first actuation of the inhalation device of FIG. 1 is shown. The means for the storage of potential energy (7) has been released from the loaded position as shown in FIG. 3. It pushes the cylinder (9) in a downstream direction such as to slide over the riser pipe (5). The interior end (5A) of the riser pipe (5) has come closer to the inlet check valve (4) which is now closed. As a result, the pressure inside the pumping chamber (3) rises and keeps the inlet valve (4) closed, but opens outlet valve (8). Liquid (F) flows from the riser pipe (5) through its exterior end (5B) towards nozzle (6).

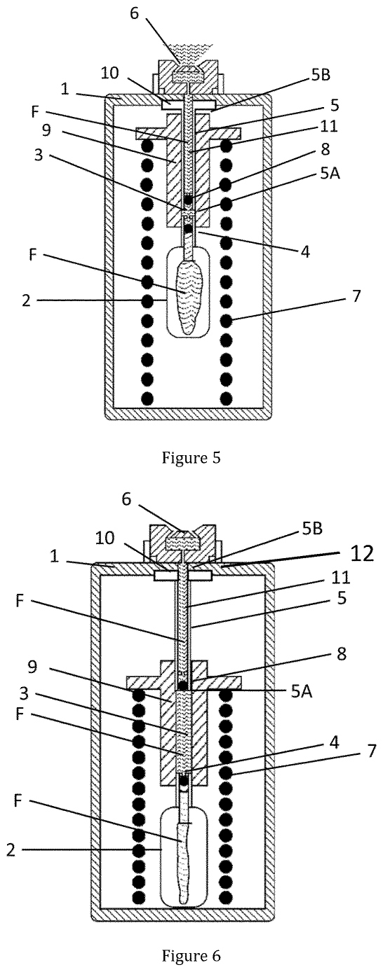

FIG. 5 shows the device of FIG. 1 in the situation at the end of the aerosol emission phase. The means for the storage of potential energy (7) is in its most relaxed end position (spring fully extended). Also, the hollow cylinder (9) has been pushed almost entirely onto riser pipe (5) such that the interior volume of pumping chamber (3) has reached its minimum. Most of the liquid (F) previously contained in the pumping chamber (3) has passed outlet valve (8) into the main segment of the riser pipe (5). Some liquid (F) has been pushed towards, and though, nozzle (6), where nebulization takes place, such that a nebulized aerosol is emitted towards the user or patient.

In FIG. 6, the device of FIG. 1 in the situation after re-filling the pumping chamber is depicted. The hollow cylinder (9) has been moved (repulsively) in an upstream direction, thus increasing the volume of the pumping chamber (3) provided by the cylinder (9). The means for the storage of potential energy (7) has been loaded (spring compressed). During movement of cylinder (9) away from the nozzle (6), a negative pressure has been generated in the pumping chamber (3), closing outlet valve (8) and opening the inlet check valve (4). As a result, further liquid (F) is drawn from reservoir (2) into the pumping chamber (3). The inhalation device's pumping chamber (3) is filled again and ready for the next ejection of liquid (F) by releasing the spring.

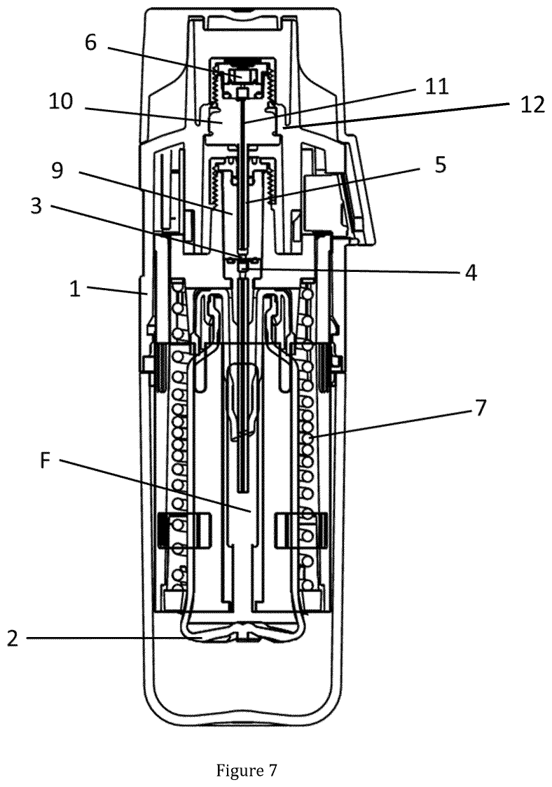

FIG. 7 depicts a further embodiment of an inhalation device according to the present invention wherein the same reference numbers are assigned to the corresponding structures as in the embodiment of FIGS. 1 to 6. As can be seen, riser pipe (5) with fluid channel (11), again, is provided in the form of a one-piece unit with holder (10). In this particular embodiment, riser pipe (5) and holder (11) in the form of a one-piece unit are made of polyether ether ketone (PEEK).

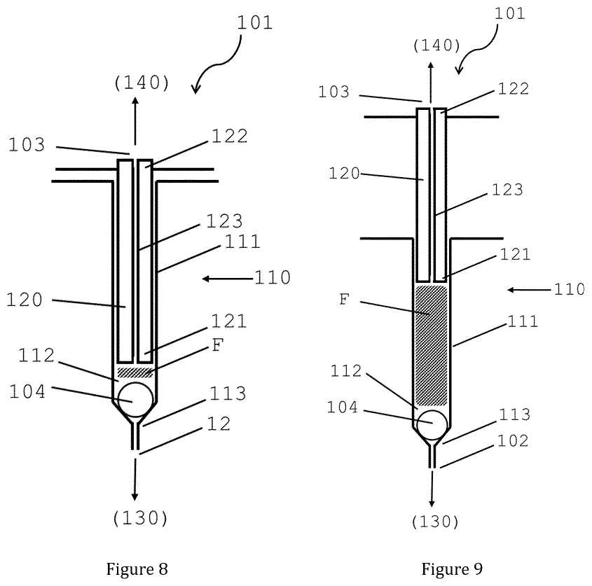

FIGS. 8 to 10 depict a schematic and not to scale representation of the second particular embodiment of the invention, utilizing a piston made from an injection moldable material in a present high-pressure medical pumping device (101) for providing discrete portions of a medical fluid (F) in pressurized form, wherein the pumping device (101) comprises a fluid inlet (102), a fluid outlet (103), and a pumping unit (110) fluidically connecting the fluid inlet (102) with the fluid outlet (103). The pumping unit is adapted to generate the discrete portions of the medical fluid (F) in pressurized form and to convey the discrete portions of the medically active fluid (F) (in a downstream direction) from the fluid inlet (102) to the fluid outlet (103). The pumping unit (110) comprises a pump cylinder (111) comprising a pump chamber (112) located within the pump cylinder (111). The pump chamber (112) comprises a fluid opening (113). The high-pressure medical pumping device (101) further comprises a piston (120) comprising a first end (121) and a second end (122) located opposite of the first end, wherein the piston (120) is at least partly located within the pump chamber (112) of the pump cylinder (111) such that at least the first end (121) of the piston is located within the pump chamber (112). The piston (120) and the pump cylinder (111) are arranged for back-and-forth stroke movement of at least one of the piston (120) and the pump chamber (112) relative to each other.

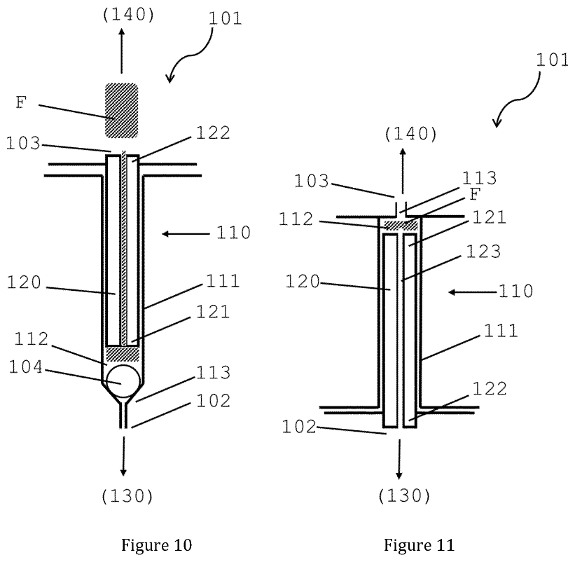

The piston (120) comprises a fluid channel (123) fluidically connecting the first end (121) of the piston with the second end (122) of the piston and being adapted to convey the medical fluid (F) from the first (121) or second end (122) of the piston to the corresponding opposite end of the piston. In the embodiment shown in FIGS. 8 to 10 the pumping unit (110) of the high-pressure medical pumping device (101) further comprises a check valve (104) located in the pump chamber close to and downstream of the fluid opening of the pump chamber (113). As can be seen in FIG. 8 in connection with FIGS. 9 and 10 as well as FIGS. 11 to 13 as described in further detail below, the pumping unit (110) of the present high-pressure medical pumping device is adapted to provide the discrete portions of the medical fluid (F) in pressurized form at a pressure of at least about 100 bar. According to the invention the piston (120) comprises an injection-moldable polymeric material.

In the embodiment shown in FIGS. 8 to 10, the fluid inlet (102) of the high-pressure medical pumping device may be connected to, for example, a reservoir (130, not shown) and the fluid outlet (103) may be connected to, for example, a nozzle (140, not shown). This allows for the pumping of discrete portions of pressurized medical fluid (F) from the fluid inlet (102) in a downstream direction towards the fluid outlet (103).

FIG. 8 depicts the status of the high-pressure medical pumping device (101) at the starting point of one pumping cycle. In this situation, piston (120) is maximally introduced into the pump chamber (112) of pump cylinder (111) resulting in minimal volume of pump chamber (112).

FIG. 9 depicts the same high-pressure medical pumping device (101) as depicted in FIG. 8, however at a subsequent status or situation of the pumping cycle. FIG. 9 shows the situation of the high-pressure medical pumping device (101) after a (repulsive) movement of the piston (120) and/or the pump cylinder relative to each other resulting in an outward movement of the piston (120) out of the pump cylinder (111) (i.e. in a downstream direction). For the avoidance of doubt, such repulsive relative movement can be implemented by a corresponding movement of the piston (120) alone or of the pump cylinder (111) alone or by a corresponding simultaneous movement of the piston (120) and the pump cylinder (111). As can be seen in FIG. 9, by this relative repulsive movement medical fluid (F) is conveyed or pumped, for example from a reservoir (130, not shown), via fluid inlet (102), fluid opening of the pump cylinder (113) and check valve (104) into pump chamber (112).

FIG. 10 depicts the same high-pressure medical pumping device (101) as depicted in FIGS. 8 and 9, however at a subsequent status or situation of the pumping cycle. FIG. 10 shows the situation of the high-pressure medical pumping device (101) after a propulsive relative movement of the piston (120) and/or the pump cylinder (111) relative to each other resulting in an inward movement of the piston (120) back into the pump cylinder (111) (i.e. in an upstream direction). For the avoidance of doubt, such propulsive relative movement can be implemented by a corresponding movement of the piston (120) alone or of the pump cylinder (111) alone or by a corresponding simultaneous movement of the piston (120) and the pump cylinder (111). As can be seen in FIG. 10, by this relative propulsive movement a discrete portion of the medical fluid (F) is conveyed or pumped from pump chamber (112) via fluid channel (123) of the piston towards fluid outlet (103) (which may, for example, be fluidically connected to a nozzle (140, not shown). As a result (and after ejection of the discrete portion of pressurized medical fluid (F) the high-pressure medical pumping device (101) is ready for the next pumping cycle.

FIGS. 11 to 13 show an alternative embodiment of the present high-pressure medical pumping device (101) which differs from the embodiment of FIGS. 8 to 10 in that the fluid inlet (102) (optionally fluidically connected to a reservoir (130, not shown) is located adjacent to the second end of the piston (122) and the fluid outlet (103) is located adjacent to the fluid opening of the pump chamber (113) (optionally fluidically connected to a nozzle (140, not shown).

FIG. 11 depicts the status of this alternative embodiment of the high-pressure medical pumping device (101) at the starting point of one pumping cycle. In this situation, piston (120) is maximally introduced into the pump chamber (112) of pump cylinder (111) resulting in minimal volume of pump chamber (112). It should be noted, that in this embodiment, the high-pressure medical pumping device (101) does not comprise a check valve, however, such check valve may be implemented, for example, at the first (121) or second end (122) of the piston or in the fluid channel of the piston (123) or at the fluid inlet (102).

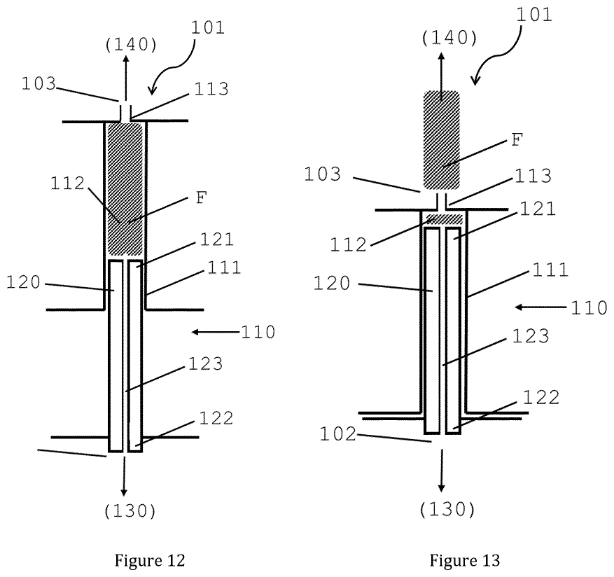

FIG. 12 depicts the same high-pressure medical pumping device (101) as depicted in FIG. 11, however, at a subsequent status or situation in the pumping cycle. FIG. 12 shows the situation of the high-pressure medical pumping device (101) after a (repulsive) movement of the piston (120) and/or the pump cylinder relative to each other resulting in an outward movement of the piston (120) out of the pump cylinder (i.e. in an upstream direction). For the avoidance of doubt, such repulsive relative movement can be implemented by a corresponding movement of the piston (120) alone or of the pump cylinder (111) alone or by a corresponding simultaneous movement of the piston (120) and the pump cylinder (111). As can be seen in FIG. 12, by this relative repulsive movement medical fluid (F) is conveyed or sucked from a reservoir (130, not shown) via fluid inlet (102) and fluid channel of the piston (123) into pump chamber (112).

FIG. 13 depicts the same high-pressure medical pumping device (101) as depicted in FIGS. 11 and 12, however at a subsequent status or situation in the pumping cycle. FIG. 13 shows the situation of the high-pressure medical pumping device (101) after a propulsive relative movement of the piston (120) and/or the pump cylinder relative to each other resulting in an inward movement of the piston (120) back into the pump cylinder (i.e. in the downstream direction). For the avoidance of doubt, such propulsive relative movement can be implemented by a corresponding movement of the piston (120) alone or of the pump cylinder (111) alone or by a corresponding simultaneous movement of the piston (120) and the pump cylinder (111). As can be seen in FIG. 13, by this relative propulsive movement a discrete portion of the medical fluid (F) is conveyed or pumped from pump chamber (112) via fluid opening of the pump cylinder (113) towards fluid outlet (103) (which may, for example, be fluidically connected to a nozzle (140, not shown). As a result (and after ejection of the discrete portion of pressurized medical fluid (F) the high-pressure medical pumping device is ready for the next pumping cycle.

### LIST OF REFERENCES

1 Housing
2 Fluid reservoir, reservoir
3 Pumping chamber
4 Inlet valve
5 Riser pipe
5A Interior end
5B Exterior end
6 Nozzle
7 Means for storing potential energy, means
8 Outlet valve
9 Hollow cylinder, cylinder
10 Holder
11 Fluid channel (of the riser pipe)
12 Receptacle
101 High-pressure medical pumping device
102 Fluid inlet
103 Fluid outlet
104 Check valve
110 Pumping unit
111 Pump cylinder
112 Pump chamber
113 Fluid opening of the pump chamber
120 Piston
121 First end of the piston
122 Second end of the piston
123 Fluid channel of the piston
130 Reservoir
140 Nozzle
150 Drive unit
160 Housing
170 Holding structure
100 Mobile inhalation device
F Liquid, fluid

1. A hand-held inhalation device for delivering a medically active fluid (F) in aerosolized form for inhalation therapy, comprising
(a) a housing (1) having a user-facing side;
(b) a nozzle (6) for generating the nebulized aerosol by collision of at least two liquid jets, the nozzle (6) being firmly affixed to the user-facing side of the housing (1) such as to be immobile relative to the housing (1);
(c) a fluid reservoir (2) arranged within the housing (1) for holding the medically active fluid; and
(d) a pumping unit arranged within the housing (1), the pumping unit having
an upstream end that is fluidically connected to the fluid reservoir (2); and
a downstream end that is fluidically connected to the nozzle (6);
wherein the pumping unit is adapted for pumping the medically active fluid (F) from the fluid reservoir (2) to the nozzle (6);
wherein the pumping unit further comprises
(i) a riser pipe (5) having an upstream end and a downstream end, wherein the riser pipe (5) is adapted to function as a piston in the pumping unit;
(ii) a holder (10) located downstream of the riser pipe being firmly affixed to the user-facing side of the housing (1) such as to be immobile relative to the housing (1) for holding the riser pipe;
(iii) a hollow cylinder (9) located upstream of the riser pipe (5), wherein the upstream end of the riser pipe (5) is inserted in the cylinder (9) such that the cylinder (9) is longitudinally movable on the riser pipe (5); and
(iv) a lockable means for storing potential energy (7) when locked and for releasing the stored energy when unlocked, the means (7) being arranged outside of, and mechanically coupled to, the cylinder (9) such that unlocking the means (7) results in a propulsive longitudinal movement of the cylinder

(9) towards the downstream end of the pumping unit;
wherein the holder (10) and the riser pipe (5) are a one-piece unit.

2. The inhalation device according to claim 1, wherein the holder (10) and the riser pipe (5) comprise an injection-moldable polymeric material, optionally at least one thermoplastic injection-moldable polymeric material.

3. The inhalation device according to any one of the preceding claims, wherein the holder (10) and the riser pipe (5) comprise the same injection-moldable polymeric material.

4. The high-pressure medical pumping device according to claim 2 or 3, wherein the injection-moldable polymeric material is selected from the group consisting of polyoxymethylene (POM), polyether ether ketone (PEEK), and polyphenylene oxide (PPO/PPE), preferably PEEK.

5. The high-pressure medical pumping device according to any one of claims 2 to 4, wherein the injection-moldable polymeric material is a mixture or blend comprising at least one injection-moldable material selected from the group consisting of PEEK, PPO/PPE and POM and at least one further injection-moldable polymeric material selected from the group consisting of acrylonitrile butadiene styrenes (ABS), acrylonitrile styrene acrylate (ASA), styrene acrylonitrile copolymer (SAN), polyacrylic acids, (such as poly(methyl methacrylate (PMMA)), acrylonitrile butadiene styrene (ABS), polyamides, polylactic acids (polylactide, PLA), polybenzimidazoles (PBI), polycarbonates (PC), polyether sulfone (PES), polyoxymethylenes (POM), polyether ether ketone (PEEK), polyetherimide (PEI), polyethylenes (PE), such as ultra-high-molecular-weight polyethylene (UHMWPE), high-density polyethylene (HDPE), medium-density polyethylene (MDPE), low-density polyethylene (LDPE)), polyphenylene oxide (PPO), polyphenylene sulfide (PPS), polypropylene (PP), polystyrene (PS), polyvinyl chloride (PVC), polyvinylidene fluoride (PVDF) and polytetrafluorethylene (PTFE).

6. The high-pressure medical pumping device according to any one of claims 2 to 5, wherein the at least one injection-moldable material is polyether ether ketone (PEEK).

7. The high-pressure medical pumping device according to any one of the preceding claims, wherein the one-piece unit comprising the riser pipe (5) and the holder (10) comprises a composite material comprising an injection-moldable material.

8. The high-pressure medical pumping device according to any one of the preceding claims, wherein the riser pipe essentially consists of the injection-moldable material.

9. The inhalation device according to any one of the preceding claims, wherein the riser pipe comprises a fluid channel fluidically connecting the upstream end of the riser pipe with the downstream end of the riser pipe for conveying the medically active fluid, optionally, wherein the fluid channel has a diameter selected within the range of from about 0.1 mm to about 1 mm.

10. The inhalation device according to any one of the preceding claims, wherein the riser pipe has a diameter selected within a range of from about 1 mm to about 3 mm.

11. The inhalation device according to any one of the preceding claims, wherein the riser pipe has a length selected within a range of from about 10 mm to about 30 mm.

12. The inhalation device according to any one of the preceding claims, wherein the nozzle comprises an impingement-type nozzle.

13. The inhalation device according to any one of the preceding claims, wherein the pumping unit is a high-pressure pumping unit adapted to expel fluid (F) at a pressure of at least 50 bar.

14. The inhalation device according to any one of the preceding claims, wherein the means for storing potential energy (7) is a spring having a load of at least 40 N in a deflected state.

15. The inhalation device according to any one of the preceding claims, being adapted to deliver the nebulized medically active aerosol discontinuously in the form of discrete units, wherein one unit is delivered per pumping cycle, optionally wherein a unit of the aerosol contains from 2 μL to 150 μL of liquid phase.

16. A hand-held inhalation device for delivering a medically active fluid (F) in aerosolized form for inhalation therapy, comprising
(a) a housing (1) having a user-facing side, the housing further comprises a receptacle configured for attaching a riser pipe (5);
(b) a nozzle (6) for generating the nebulized aerosol by collision of at least two liquid jets, the nozzle (6) being firmly affixed to the user-facing side of the housing (1) such as to be immobile relative to the housing (1);
(c) a fluid reservoir (2) arranged within the housing (1) for holding the medically active fluid; and
(d) a pumping unit arranged within the housing (1), the pumping unit having
an upstream end that is fluidically connected to the fluid reservoir (2); and
a downstream end that is fluidically connected to the nozzle (6);
wherein the pumping unit is adapted for pumping the medically active fluid (F) from the fluid reservoir (2) to the nozzle (6);
wherein the pumping unit further comprises
(i) the riser pipe (5) monolithically comprising:
a riser pipe segment having an upstream end and a downstream end
a holder segment (10) located at the downstream end of the riser pipe segment, the holder segment attached to the receptacle of the housing and firmly affixing the riser pipe (5) to the user-facing side of the housing (1) such as to be immobile relative to the housing (1);
(ii) a hollow cylinder (9) located upstream of the riser pipe (5), wherein the upstream end of the riser pipe (5) is inserted in the cylinder (9) such that the cylinder (9) is longitudinally movable on the riser pipe segment; and
(iii) a lockable means for storing potential energy (7) when locked and for releasing the stored energy when unlocked, the means (7) being arranged outside of, and mechanically coupled to, the cylinder (9) such that unlocking the means (7) results in a propulsive longitudinal movement of the cylinder (9) towards the downstream end of the pumping unit.
